SEQUENCE LISTING (4 sequences)

SEQ ID NO: 1 - protein, 314 aa, Homo sapiens
MRIAVICFCLLGITCAIPVKQADSGSSEEKQLYNKYPDAVATWLNPDPSQKQNLLAPQNAVSSEETNDFKQETLPSKSNESHDHMDDMDDEDDDDHVDSQDSIDSNDSDDVDDTDDSHQSDESHHSDESDELVTDFPTDLPATEVFTPVVPTVDTYDGRGDSVVYGLRSKSKKFRRPDIQYPDATDEDITSHMESEELNGAYKAIPVAQDLNAPSDWDSRGKDSYETSQLDDQSAETHSHKQSRLYKRKANDESNEHSDVIDSQELSKVSREFHSHEFHSHEDMLVVDPKSKEEDKHLKFRISHELDSASSEVN

SEQ ID NO: 2 - protein, 168 aa, Homo sapiens
MRIAVICFCLLGITCAIPVKQADSGSSEEKQLYNKYPDAVATWLNPDPSQKQNLLAPQNAVSSEETNDFKQETLPSKSNESHDHMDDMDDEDDDDHVDSQDSIDSNDSDDVDDTDDSHQSDESHHSDESDELVTDFPTDLPATEVFTPVVPTVDTYDGRGDSVVYGLR

SEQ ID NO: 3 - protein, 167 aa, Homo sapiens
MRIAVICFCLLGITCAIPVKQADSGSSEEKQLYNKYPDAVATWLNPDPSQKQNLLAPQNAVSSEETNDFKQETLPSKSNESHDHMDDMDDEDDDDHVDSQDSIDSNDSDDVDDTDDSHQSDESHHSDESDELVTDFPTDLPATEVFTPVVPTVDTYDGRGDSVVYGL

SEQ ID NO: 4 - protein, 166 aa, Homo sapiens
MRIAVICFCLLGITCAIPVKQADSGSSEEKQLYNKYPDAVATWLNPDPSQKQNLLAPQNAVSSEETNDFKQETLPSKSNESHDHMDDMDDEDDDDHVDSQDSIDSNDSDDVDDTDDSHQSDESHHSDESDELVTDFPTDLPATEVFTPVVPTVDTYDGRGDSVVYG

(19) United States
(12) Patent Application Publication
(10) Pub. No.: US 2019/0367602 A1
(43) Pub. Date: Dec. 5, 2019

(54) NOVEL MONOCLONAL ANTIBODIES TO OSTEOPONTIN

(71) Applicant: Synergenics, LLC, San Francisco, CA (US)

(72) Inventors: John P. MCKEARN, St. Louis, MO (US); Jeremy BLITZER, San Francisco, CA (US)

(21) Appl. No.: 16/468,634

(22) PCT Filed: Dec. 12, 2017
(86) PCT No.: PCT/US17/65778

§ 371 Date: Jun. 11, 2019

Related U.S. Application Data
(60) Provisional application No. 62/432,791, filed on Dec. 12, 2016.

(51) Int. Cl. C07K 16/24; C12N 5/0781; G01N 33/68

(52) U.S. Cl. CPC C07K 16/24; C12N 5/0635; G01N 33/6893; C07K 2317/76; C07K 2317/24; C07K 2317/34; C07K 2317/33; C07K 2317/31; C07K 2317/14

(57) ABSTRACT

The present disclosure relates to the development of antibodies that are targeted to disease specific function directing regions of osteopontin. Such antibodies are capable of binding osteopontin and of selectively blocking one or more functions of osteopontin. In one aspect, the disclosures are based on the discovery that such antibodies spontaneously occur in certain diseases, and have therapeutic utility for the treatment of one or more osteopontin related diseases, where they are capable of selectively blocking the role of osteopontin in disease progression.

### CROSS REFERENCE TO RELATED APPLICATIONS

This application claims the benefit of U.S. Provisional Application No. 62/432,791, filed Dec. 12, 2016; the disclosure of which is hereby incorporated by reference in its entirety.

### INCORPORATION OF SEQUENCE LISTING

The sequence listing that is contained in the file named “SYN0001401PC_ST25,” which is 7.28 kilobytes as measured in Microsoft Windows operating system and was created on Dec. 12, 2017, is filed electronically herewith and incorporated herein by reference.

### TECHNICAL FIELD

The present disclosure relates to methods for obtaining novel high affinity auto-antibodies that selectively bind to osteopontin in a disease selective fashion, methods for obtaining immortalized B cells producing such antibodies, and methods for using the antibodies to treat osteopontin related diseases and disorders such as arthritis.

### BACKGROUND

Osteopontin, also known as OPN (Oldberg et al. (1986) Proc. Natl. Acad. Sci. USA 83:8819), 2ar (Smith and Denhardt (1987) J Cell Biochem. 34(1):13-22), transformation-associated secreted phosphoprotein (Senger et al. (1989) Anticancer Res. 48:1291), secreted phosphoprotein 1, (SPP1), bone sialoprotein, urinary stone protein (BSPI), MGC110940 and early T-lymphocyte activation-1 (Eta-1) (Patarca et al. (1991) Proc. Natl. Acad. Sci. USA 88:2736) is a multifunctional secreted glycoprotein.

Osteopontin is expressed by a wide variety of cell types including bone (Oldberg et al. (1986) Proc. Natl. Acad. Sci. USA 83:8819; Oldberg et al. (1986) J. Biol. Chem. 263:19433-19436), smooth muscle cells (e.g., cells of the vascular system) (Giachelli et al. (1991) Biochem. Biophys. Res. Commun. 177: 867-873), activated T-lymphocytes (Patarca et al. (1989) J. Exp. Med. 170:145-161; Patarca et al. (1991) Proc. Natl. Acad. Sci. USA, supra), macrophages (Singh et al. (1990), J. Exp. Med 171:1931-1942), and carcinomas and sarcomas (Senger et al., supra). In other tissues, osteopontin is expressed during various developmental stages and circulating levels of the protein have been found to be elevated in individuals with autoimmune diseases. Osteopontin is induced by oxidative stress, including ischemia/reperfusion, heat shock or starvation, and exerts antioxidant effects by down-regulation of inducible nitric oxide synthetase (conferring protection against killing by macrophages). Osteopontin is also elevated in sera from patients with advanced metastatic cancer and cellular transformation may lead to enhanced osteopontin expression and increased metastatic activity.

In humans, the single gene encoding osteopontin is located on the long arm of chromosome 4. The osteopontin mRNA transcript includes seven exons, six of which are translated (exon 1 is not translated), and three splice variants have been identified: osteopontin-a (OPN-a) mRNA contains all seven exons, osteopontin-b (OPN-b) mRNA lacks exon 5, and osteopontin-c (OPN-c) mRNA lacks exon 4 (Saitoh et al., Lab. Invest. 72:55-63, 1995). Integrin binding sites are located in a central part of the protein and are primarily encoded by exon 6. The osteopontin receptor, CD44, binds the C-terminus. Numerous studies have suggested that various osteopontin splice variants have differential roles in various cancers, including that of breast (He et al., Oncogene 25: 2192-2202, (2006); Mirza et al., Int. J. Cancer 122, 889-897, (2008)), pancreas (Sullivan et al., Surgery 146: 232-240, (2009)), and hepatocellular carcinoma (Chae et al. Int. J. Oncol. 35: 1409-1416, (2009)). In particular, the OPN-c splice variant has been shown to induce anchorage-independent growth in human breast cancer (He et al., (2006)) and hepatocellular carcinoma (Chae et al., (2009)) cells, and have selective expression in breast (Mirza et al., (2008)) and pancreatic cancer (Sullivan et al., (2009)). OPN-a and OPN-b have also been shown to have pro-migratory properties on hepatocellular carcinoma cells (Chae et al., (2009)). These splice variant-specific properties of osteopontin may thus facilitate its role in the invasion and metastasis of various malignancies.

Osteopontin is subject to a large number of post-translational modifications including: thrombin cleavage, sulfation, glycosylation, trans-glutamination, and phosphorylation. Multiple phosphorylated and nonphosphorylated forms of osteopontin are secreted by cells and are differentially stimulated by tumor promoters (Kubota et al. (1989) Biochem. Biophys. Res. Commun. 162: 1453-1459). The phosphorylation of osteopontin appears to be an important regulatory mechanism, and osteopontin has at least 58 consensus phosphorylation sites for different types of kinases which are organized in eight clusters. These include 9 potential phosphorylation sites for either casein kinase I, casein kinase II or mammary gland casein kinase, in addition to potential phosphorylation sites for cAMP- and cGMP-dependent protein kinases, calmodulin-dependent protein kinase, and protein kinase C (US20070134229A1).

Osteopontin contains several integrin binding sites and includes the integrin binding motif, Gly-Arg-Gly-Asp-Ser (“RGD” or “GRGDS” motif), just N-terminal to a thrombin cleavage site, which is involved in cell attachment and spreading via interactions via alpha V integrins including αᵥβ₃, αᵥβ₁, αᵥβ₅, α₉β₁ and α₄β₁. Cleavage of osteopontin with thrombin creates a truncated 168 amino acid form of osteopontin, termed osteopontin-R, which exposes a cryptic epitope, Ser-Val-Val-Tyr-Gly-Leu_Arg (SVVYGLR) which provides additional cryptic binding sites for α₉β₁, α₉β₄, α₄β₄ and α₄β₁ integrins. Further proteolysis of this protein by carboxypeptidase B can remove the C-terminal arginine (R) residue to produce the 167 amino acid form of osteopontin, termed osteopontin-L. Additional proteolysis of osteopontin-L by matrix metalloproteinases during inflammatory processes can also create a 166 amino acid form of osteopontin (“osteopontin-166”) by removal of the C-terminal leucine in osteopontin-L. While the 166 and 167 amino acid length forms of osteopontin are structurally similar, the deletion of the final amino acid in osteopontin L creates a new protein form with significantly altered conformation, and protein-protein binding specificity. Specifically, osteopontin-166 binds to a unique set of proteins, including the integrins, αvβ1, αvβ3, αvβ5, and α5β1, compared to the osteopontin-L, osteopontin-R and full-length forms of osteopontin.

A distinct receptor-ligand interaction between CD44 and the C-terminal region of osteopontin has also been shown to play a role in mediating chemotaxis and/or cell or attachment. Osteopontin also includes an Asp-rich sequence (ELVTDFTDLPAT), reported to bind α₄β₁ integrin and a C-terminal heparin-binding domain capable of binding fibronectin, and vitronectin.

As its name suggests, osteopontin was originally implicated in calcium homeostasis (e.g., calcification), bone remodeling, osteoporosis and osteoclast function. It is frequently found in pathological calcifications such as atherosclerotic plaques, sclerotic glomeruli, ecotopic calcification and kidney stones.

Additionally the Osteopontin gene is expressed in T cells early in the course of bacterial infections (within 48 hours), and interaction of its protein product with macrophages can induce inflammatory responses. Genetic resistance to infection by certain strains of Rickettsia may depend on osteopontin-dependent attraction of monocytes into infectious sites and acquisition of bacteriocidal activity. Furthermore, the granulomatous responses characteristic of sarcoidosis and tuberculosis are associated with high levels of osteopontin expression.

Osteopontin has also been implicated in various other events that are important to cell-mediated immunity. For example, osteopontin is associated with monocyte-macrophage differentiation, giant cell formation and the inhibition of apoptosis in various cell types, including pro-B cells. Osteopontin also inhibits nitric oxide production by macrophages and has been associated with tissue repair, fibrosis and dystrophic calcification after immunological injury.

Osteopontin is present in multiple sclerosis lesions, and the administration of recombinant osteopontin to an experimental relapsing-remitting model of MS, experimental autoimmune encephalomyelitis (EAE), results in a rapid induction of relapse. (Nat. Rev. Imm. 15 May 2009 doi:10.1038/nri2548). Administration of osteopontin triggers neurological relapse by two mechanisms. First, osteopontin stimulates the expression of pro-inflammatory mediators, including T helper 1 (T_{H}1)- and (T_{H}17)-type cytokines in myelin-specific T cells, simultaneously; osteopontin inhibits forkhead box O3A (FOXO3A) dependent apoptosis of autoreactive immune cells. The net result of these actions is the osteopontin mediated survival of autoreactive T cells. In this model blocking α4β₁ integrin lead to an inhibition of this relapse, suggesting that osteopontin mediates this effect in least in part through interactions with α₄β₁ integrin. Additionally, thrombin mediated cleavage of osteopontin in situ leads to the exposure of at least two additional cryptic binding sites for α₄β₁ integrin in the cleaved forms of osteopontin, as described above further enhancing neurological relapse.

Therefore, the present disclosure also relates to the use of antibodies to selectively target the disease selective forms of osteopontin, that are formed in situ in multiple sclerosis, and the use of such antibodies for treating and preventing the development of demyelinating diseases of the CNS or PNS, including neuropathies and neurodegenerative diseases, as well as autoimmune reactions associated with traumatic nerve injury and stroke.

Osteopontin mediates effects on cell mediated immunity by enhancing Th1, and inhibiting Th2 cytokine expression. It directly induces macrophages to induce the production of IL-12, and inhibits IL-10 production. Osteopontin also costimulates T cell proliferation. Osteopontin increases CD3-mediated T-cell production of interferon gamma and CD40 ligand, which augments T-cell dependent IL-12 production by human monocytes. Osteopontin can also induce proliferation of B-cells and antibody production through the involvement of CD40L of B cells.

Factors augmenting Th1 and inhibiting Th2 cytokine expression (IL-10, IL-4, IL-5) function as powerful modulators of cell-mediated immunity. The development of cell-mediated (type-1) immune responses is necessary for protection against the growth of many infectious pathogens and can mediate autoimmune host tissue destruction. An essential early step in macrophage activation by microbial pathogens and foreign body reactions is macrophage production of IL-12 at sites of infection, whereas early IL-10 production inhibits this response. Although IL-12 responses can be triggered by an interaction between the CD40 ligand on activated T cells and CD40 on macrophages, this interaction also induces the inhibitory IL-10 cytokine, and its transient nature may not suffice for sustained IL-12 induction in vitro or in vivo.

Osteopontin is chemoattractant and supports adhesion of human and murine T cells and macrophages in vitro. In vivo, macrophages accumulate at sites of subcutaneous injection of Osteopontin. In addition, Osteopontin may directly induce chemotaxis and indirectly facilitate macrophage migration to other chemoattractants. In vitro migratory and adhesive effects of Osteopontin are mediated by RGD-dependent (integrin) and RGD-independent (CD44) receptors. Osteopontin deficient mice exhibit a five-fold reduction in macrophage infiltration compared with wild-type controls following renal injury. Further, subcutaneous injection of polyvinylpyrrolidone (PVP) induces macrophage-rich granulomas and cellular accumulation after PVP injection is markedly reduced in Osteopontin deficient mice.

Given the central role of osteopontin in regulating T cell chemotaxis and cell mediated immunity, a number of inflammatory diseases and disorders are therefore susceptible to treatment in accordance with the present disclosure, including but not limited to chronic asthma, atherosclerosis, restenosis, ischemia/reperfusion, arthritis, inflammatory bowel disease, ulcerative colitis, type 1 diabetes, systemic lupus erythematosis, rheumatoid arthritis, osteoarthritis, and multiple sclerosis.

Monoclonal antibodies against osteopontin may have use in the treatment of inflammation, fibrosis, vascular occlusion and scarring that occur with the progression of any of these autoimmune diseases as well as chronic infections such as tuberculosis and sarcoidosis.

While osteopontin has been shown to have a recently identified pro-inflammatory role in Th1-mediated immunity, it also appears to have anti-inflammatory effects in different pathological responses. Osteopontin appears to have a role in aberrant tissue repair, has been associated with ras oncogenic transformation of cells (Wu et al. 2000, Brit. J. Cancer, 83: 156-163) and is involved in interactions with CD44 and the formation of metastasis (Weber et al., 1997. Proc. Assoc. Amer. Phys. 109: 1-9). Osteopontin has also been shown to be secreted by malignant tumors and is believed to play an important role in metastasis formation.

Therefore, osteopontin appears to have multiple functions, both in normal physiology and in various disease pathologies. The multifunctional nature of osteopontin reflects the dynamic temporal and spatial interplay of multiple expressed isoforms (e.g., splice variants), complex post-translation modifications, different conformational and proteolytic forms, and multiple interacting partners of osteopontin.

Given the important role that osteopontin plays in cellular processes including cell attachment, cell spreading and chemotaxis as well as the important functions it has in diverse processes including wound healing, tissue remodeling, cell mediated immunity, autoimmunity, the immune response, bone development, and metastasis, there exists a need to identify antibodies that can selectively inhibit one or more of the unique functions of osteopontin. In particular, there exists a need for identifying antibodies that are safe and well tolerated in man, and that can selectively block one or more functions of disease selective forms of osteopontin and can be used to treat or prevent the severity or recurrence of an osteopontin related disease.

The present disclosure is based, at least in part, on the discovery and identification of auto-antibodies to osteopontin that occur naturally in subjects with osteoarthritis, rheumatoid arthritis, and multiple sclerosis and which surprisingly have utility for the treatment and prevention of osteopontin related diseases.

It has been further discovered that these antibodies are targeted to specific domains of the naturally-occurring protein, and can selectively block the pro-inflammatory functions of osteopontin within disease tissues.

The development of auto-antibodies to these biologically active domains and forms of osteopontin in situ, has led to the development of new therapeutic antibodies that can selectively block a subset of osteopontin's biological functions and are useful for the treatment of various osteopontin-related diseases, including osteoarthritis, rheumatoid arthritis and multiple sclerosis.

Importantly therapeutic human auto-antibodies have unique properties and advantages compared to more traditional antibodies to osteopontin that have been created from recombinant osteopontin, or prepared from classical antibody discovery and optimization technologies such as mouse immunization, CDR grafting, phage display or in vitro affinity maturation of germline antibodies. These properties include i) improved safety and cross reactivity profiles that stem from the use of fully human antibodies that have undergone natural clonal selection and affinity maturation in vivo, ii) improved affinity and potency to the active form (conformer) of osteopontin actually present in situ within a specific disease state iii) improved targeting to the functionally significant regions or interaction domains of the protein (function directing regions) to selectively modulate a specific function of osteopontin associated with a disease state, while reducing the side effects associated with broadly inhibiting osteopontin activity.

### SUMMARY OF THE DISCLOSURE

In general terms, the current disclosure is concerned with the development of antibodies that are targeted to disease specific function directing regions of osteopontin. Such antibodies are capable of binding osteopontin and of selectively blocking one or more functions of osteopontin. In one aspect, the disclosures is based on the discovery that such antibodies spontaneously occur in certain diseases, and have therapeutic utility for the treatment of one or more osteopontin related diseases, where they are capable of selectively blocking the role of osteopontin in disease progression.

In one aspect, the present disclosure includes a method for selecting a therapeutic antibody to osteopontin comprising the steps of; screening a subject with an osteopontin related disease, for osteopontin specific antibodies, and isolating or amplifying one or more nucleic acids from the subject's B cells encoding an antibody heavy or light chain, or portion thereof, that binds to osteopontin. In one aspect, of this method the one or more nucleic acids obtained from the subject's B cells is obtained by PCR amplification of isolated B cells. In one aspect, of this method, the isolated B cells are immortalized, cultured and screened prior to isolation or amplification of the nucleic acids. In one aspect, the subject is a human. In another aspect the human has an osteopontin related disease selected from the group consisting of osteoarthritis, multiple sclerosis and rheumatoid arthritis.

In another aspect, the present disclosure includes a method for selecting a human therapeutic antibody to osteopontin comprising the steps of; a)isolating memory B cells obtained from a human donor with osteoarthritis, multiple sclerosis or rheumatoid arthritis; b) immortalizing the cells isolated in step a) to produce memory B cell cultures; c) screening the memory B cell cultures obtained in step b) for cultures producing osteopontin specific antibodies; and d)isolating or amplifying one or more nucleic acids from the memory B cell cultures producing osteopontin specific antibodies of step c) encoding an antibody heavy or light chain, or portion thereof, that binds to a function directing region of osteopontin.

In another aspect the present disclosure includes method for selecting a human therapeutic antibody for treating an osteopontin related disease comprising the steps of; a) isolating memory B cells obtained from a human donor with osteoarthritis or rheumatoid arthritis; b) immortalizing the cells isolated in step a) to produce memory B cell cultures; c) screening the memory B cell cultures obtained in step b) for cultures producing osteopontin specific antibodies; and d)isolating or amplifying one or more nucleic acids from the memory B cell cultures producing osteopontin specific antibodies of step c) encoding an antibody heavy or light chain, or portion thereof, that binds to a function directing region of osteopontin.

In another aspect the present disclosure includes a method for selecting a human therapeutic antibody for treating multiple sclerosis comprising the steps of; a)isolating memory B cells obtained from a human donor with osteoarthritis, multiple sclerosis or rheumatoid arthritis; b) immortalizing the cells isolated in step a) to produce memory B cell cultures; c) screening the memory B cell cultures obtained in step b) for cultures producing osteopontin specific antibodies; d) isolating or amplifying one or more nucleic acids from the memory B cell cultures producing osteopontin specific antibodies of step c) encoding an antibody heavy or light chain, or portion thereof, that binds to a function directing region of osteopontin.

In another aspect, the present disclosure includes a method for selecting a human therapeutic antibody for treating rheumatoid arthritis comprising the steps of; a) isolating memory B cells obtained from a human donor with osteoarthritis, multiple sclerosis or rheumatoid arthritis; b) immortalizing the cells isolated in step a) to produce memory B cell cultures; c) screening the memory B cell cultures obtained in step b) for cultures producing osteopontin specific antibodies; d) isolating or amplifying one or more nucleic acids from the memory B cell cultures producing osteopontin specific antibodies of step c) encoding an antibody heavy or light chain, or portion thereof, that binds to a function directing region of osteopontin.

In another aspect, the present disclosure includes a method for selecting a human therapeutic antibody for treating osteoarthritis comprising the steps of; a) isolating memory B cells obtained from a human donor with osteoarthritis, multiple sclerosis or rheumatoid arthritis; b) immortalizing the cells isolated in step a) to produce memory B cell cultures; c) screening the memory B cell cultures obtained in step b) for cultures producing osteopontin specific antibodies; d)isolating or amplifying one or more nucleic acids from the memory B cell cultures producing osteopontin specific antibodies of step c) encoding an antibody heavy or light chain, or portion thereof, that binds to a function directing region of osteopontin.

In another aspect, the disclosure includes a process for preparing memory B cell clones expressing a therapeutic antibody to Osteopontin, to treat an osteopontin related disease comprising the steps of; a) isolating memory B cells obtained from a human donor with osteoarthritis, multiple sclerosis or rheumatoid arthritis; b) isolating a sample of memory B cells from the human donor, c) immortalizing the memory B cells by transforming memory B cells with Epstein Barr virus (EBV) in the presence of a polyclonal B cell activator; d) screening the memory B cells for the expression of antibodies that bind to osteopontin, and selecting memory B cells that exhibit antibodies that specifically bind to a function directing region of osteopontin.

In one aspect, of any of these methods, the subject is pre-selected to have antibody titers for osteopontin binding of greater than (>1:1000) measured via an ELISA assay.

In another aspect of any of these methods, the subject with multiple sclerosis is in stable remission. In another aspect of any of these methods, the subject with rheumatoid arthritis is in stable remission. In another aspect of any of these methods, the subject with osteoarthritis is in stable remission.

In another aspect of any of these methods, the memory B cell cultures producing osteopontin specific antibodies are sub-cloned.

In another aspect of any of these methods, the antibodies are screened for binding to an epitope within an arginine-glycine-aspartate (RGD) containing domain of osteopontin.

In another aspect of any of these methods, the antibodies that screened for binding to an epitope within a serine-valine-valine-tyrosine-glycine-leucine-arginine (SVVYGLR) containing domain of osteopontin.

In another aspect of any of these methods, the antibodies are screened for binding to an epitope within only the OPN-a splice variant of osteopontin.

In another aspect of any of these methods, the antibodies are screened for binding to an epitope within only the OPN-b splice variant of osteopontin.

In another aspect of any of these methods, the antibodies are screened for binding to an epitope within only the OPN-c splice variant of osteopontin.

In another aspect of any of these methods, the antibodies are screened for binding to osteopontin-R. In another aspect of any of these methods, the antibodies are screened for binding to osteopontin-L. In another aspect of any of these methods, the antibodies are screened for binding to osteopontin-166.

In another aspect of any of these methods, the antibodies are screened for binding to the CD44 binding site of osteopontin.

In another aspect of any of these methods, the antibodies are screened for binding to the αᵥβ₃ binding site of osteopontin. In another aspect of any of these methods, the antibodies are screened for binding to the αᵥβ₁ binding site of osteopontin. In another aspect of any of these methods, the antibodies are screened for binding to the αᵥβ₅ binding site of osteopontin. In another aspect of any of these methods, the antibodies are screened for binding to the α₅β₁ binding site of osteopontin. In another aspect of any of these methods, the antibodies are screened for binding to both the αᵥβ₃ binding site of osteopontin and the CD44 binding site osteopontin. In another aspect of any of these methods, the memory B cell subclones are further screened for cultures producing antibodies that are bind to an epitope within an ELVTDFTDLPAT containing domain of osteopontin.

In another embodiment, the present disclosure also includes a method for treating an osteopontin related disease, in a patient in need thereof, comprising the steps of: a) identifying one or more subjects suffering from osteoarthritis, multiple sclerosis or rheumatoid arthritis; wherein the subjects are characterized by the presence of anti-osteopontin neutralizing antibodies; and b) isolating B cells from the positive subjects screened in step a); c) isolating or amplifying an nucleic acid from the selected B cell of step b) encoding an antibody heavy or light chain, or portion thereof, that binds to osteopontin, d) administering the antibody to osteopontin encoded by the nucleic acid obtained in step (c) to the patient. In one aspect, of this method, the subject is a human.

In one aspect, of this method, the osteopontin related disease is selected from the group consisting of multiple sclerosis, rheumatoid arthritis, osteoarthritis, metastatic cancer, systematic lupus erythematosis or autoimmune renal disease, idiopathic fibrosis, allergic disease, hepatitis, valvular heart disease, cardiac remodeling, and ectopic tissue calcification.

In another aspect of the disclosure, the disclosure includes a human, or humanized therapeutic auto-antibody made by a method of any of the methods of the disclosure.

In another aspect of the disclosure, the disclosure includes a purified isolated human therapeutic auto antibody to osteopontin.

In another aspect, the disclosure includes a bispecific antibody that binds to two distinct domains or conformations of osteopontin. In one aspect, the bispecific antibody comprises at least one antigen-binding domain derived from a therapeutic auto antibody to osteopontin obtained using any of the methods of the disclosure. In one aspect, the bispecific antibody binds to both the αᵥβ₃ binding site of osteopontin and the CD44 binding site osteopontin.

In one aspect, at least one antigen-binding domain of the bispecific antibody binds to osteopontin-R. In another aspect, at least one antigen-binding domain of the bispecific antibody binds osteopontin-L. In another aspect, at least one antigen-binding domain of the bispecific antibody binds to osteopontin-166. In another aspect, at least one antigen-binding domain of the bispecific antibody binds to the CD44 binding site of osteopontin.

In another aspect, at least one antigen-binding domain of the bispecific antibody binds to the αᵥβ₃ binding site of osteopontin. In another aspect, at least one antigen-binding domain of the bispecific antibody binds to the αᵥβ₁ binding site of osteopontin. In another aspect, at least one antigen-binding domain of the bispecific antibody binds to the αᵥβ₅ binding site of osteopontin. In another aspect, at least one antigen-binding domain of the bispecific antibody binds to the α₅β₁ binding site of osteopontin. In another aspect, at least one antigen-binding domain of the bispecific antibody binds to both the αᵥβ₃ binding site of osteopontin and the CD44 binding site osteopontin.

In another aspect of the disclosure, the disclosure includes a pharmaceutical composition comprising a therapeutic auto antibody to osteopontin obtained using any of the claimed methods. In one aspect, the therapeutic auto antibody to osteopontin is human. In another aspect, the therapeutic auto antibody to osteopontin is a humanized non human antibody. In one embodiment, the antibody recognizes an epitope within an arginine-glycine-aspartate (RGD) containing domain of osteopontin.

In another embodiment, the antibody binds to the αᵥβ₃ binding site of osteopontin. In another embodiment, the antibody binds to the αᵥβ₁ binding site of osteopontin. In another embodiment, the antibody binds to the αᵥβ₅ binding site of osteopontin. In another embodiment, the antibody binds to the α₅β₁ binding site of osteopontin. In another embodiment, the antibody binds to the αᵥβ₃ binding site of osteopontin and the CD44 binding site osteopontin.

In one aspect, the antibody binds to osteopontin-R. In another aspect, the antibody binds osteopontin-L. In another aspect, the antibody binds to osteopontin-166. In another aspect, antibody binds to the CD44 binding site of osteopontin.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

A better understanding of the features and advantages of the present disclosure can be obtained by reference to the following detailed description that sets forth illustrative embodiments, in which the principles of the disclosure are utilized, and the accompanying drawings of which:

FIG. 1. Shows a schematic testing funnel to enable the selective identification of the claimed human auto-antibodies to osteopontin.

### DETAILED DESCRIPTION OF DISCLOSURE

### Definitions

As used herein and in the appended claims, the terms “a,” “an,” and “the” can mean, for example, one or more, or at least one, of a unit unless the context clearly dictates otherwise. Thus, for example, reference to “an antibody” includes a plurality of such antibodies and reference to “a variable domain” includes reference to one or more variable domains and equivalents thereof known to those skilled in the art, and so forth. In the event that there is a plurality of definitions for a term herein, those in this section prevail unless stated otherwise.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the disclosure. The upper and lower limits of these smaller ranges can independently be included or excluded in the range, and each range where either, neither or both limits are included in the smaller ranges is also encompassed within the disclosure, subject to any specifically excluded limit in the stated range. Where the stated range includes one or both of the limits, ranges excluding either or both of those included limits are also included in the disclosure.

The terms used in this specification generally have their ordinary meanings in the art, within the context of this disclosure and in the specific context where each term is used. Numerical quantities given herein are approximate unless stated otherwise, meaning that the terms “about” or “approximately” can be inferred when not expressly stated. Certain terms are discussed below, or elsewhere in the specification, to provide additional guidance to the practitioner in describing the compositions and methods of the disclosure and how to make and use them.

The term “antibody” describes an immunoglobulin whether natural or partly or wholly synthetically produced. The term also covers any polypeptide or protein having a binding domain which is, or is homologous to, an antigen-binding domain. CDR grafted antibodies, including bi-specific antibodies, and humanized antibodies, in which one or more of the CDRs are derived from antibodies obtained from B-cells identified, cloned, or selected using any of the methods disclosed or claimed herein are also contemplated by this term.

“Native Ig G antibodies” and “native Ig G immunoglobulins” are typically heterotetrameric glycoproteins of about 150,000 daltons, composed of two identical light (L) chains and two identical heavy (H) chains. Each light chain is, in some cases, linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (“V_{H}”) followed by a number of constant domains (“C_{H}”). Each light chain has a variable domain at one end (“V_{L}”) and a constant domain (“C_{L}”) at its other end; the constant domain of the light chain is aligned with the first constant domain of the heavy chain, and the light-chain variable domain is aligned with the variable domain of the heavy chain. Particular amino acid residues are believed to form an interface between the light- and heavy-chain variable domains.

The term “variable domain” refers to protein domains that differ extensively in sequence among family members (i.e., among different isoforms, or in different species). With respect to antibodies, the term “variable domain” refers to the variable domains of antibodies that are used in the binding and specificity of each particular antibody for its particular antigen. However, the variability is not evenly distributed throughout the variable domains of antibodies. It is concentrated in three segments called hypervariable regions both in the light chain and the heavy chain variable domains. The more highly conserved portions of variable domains are called the “framework region” or “FR.” The variable domains of unmodified heavy and light chains each comprise four FRs (FR1, FR2, FR3 and FR4, respectively), largely adopting a n-sheet configuration, connected by three hypervariable regions, which form loops connecting, and in some cases forming part of, the n-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991), pages 647 669). The constant domains are not involved directly in binding an antibody to an antigen, but exhibit various effector functions, such as participation of the antibody in antibody-dependent cellular toxicity.

The term “hypervariable region” when used herein refers to the amino acid residues of an antibody which are responsible for antigen-binding. The hypervariable region comprises amino acid residues from three “complementarity determining regions” or “CDRs,” which directly bind, in a complementary manner, to an antigen and are known as CDR1, CDR2, and CDR3, respectively.

In the light chain variable domain, the CDRs correspond to approximately residues 24-34 (CDRL1), 50-56 (CDRL2) and 89-97 (CDRL3), and in the heavy chain variable domain the CDRs correspond to approximately residues 31-35 (CDRH1), 50-65 (CDRH2) and 95-102 (CDRH3); Kabat et al., Sequences of Proteins of Immunological Interest, 5th Ed. Public Health Service, National Institutes of Health, Bethesda, Md. (1991)) and/or those residues from a “hypervariable loop” (i.e., residues 26-32 (L1), 50-52 (L2) and 91-96 (L3) in the light chain variable domain and 26-32 (H1), 53-55 (H2) and 96-101 (H3) in the heavy chain variable domain; Chothia and Lesk J., Mol. Biol. 196:901-917 (1987)).

As used herein, “variable framework region” or “VFR” refers to framework residues that form a part of the antigen binding pocket and/or groove that may contact antigen. In some embodiments, the framework residues form a loop that is a part of the antigen binding pocket or groove. The amino acids residues in the loop may or may not contact the antigen. In an embodiment, the loop amino acids of a VFR are determined by inspection of the three-dimensional structure of an antibody, antibody heavy chain, or antibody light chain. The three-dimensional structure can be analyzed for solvent accessible amino acid positions as such positions are likely to form a loop and/or provide antigen contact in an antibody variable domain. Some of the solvent accessible positions can tolerate amino acid sequence diversity and others (e.g., structural positions) can be less diversified. The three-dimensional structure of the antibody variable domain can be derived from a crystal structure or protein modeling. In some embodiments, the VFR comprises, consists essentially of, or consists of amino acid positions corresponding to amino acid positions 71 to 78 of the heavy chain variable domain, the positions defined according to Kabat et al., 1991. In some embodiments, VFR forms a portion of Framework Region 3 located between CDRH2 and CDRH3. Preferably, VFR forms a loop that is well positioned to make contact with a target antigen or form a part of the antigen binding pocket.

Depending on the amino acid sequence of the constant domain of their heavy chains, immunoglobulins can be assigned to different classes. There are five major classes of immunoglobulins: IgA, IgD, IgE, IgG, and IgM, and several of these can be further divided into subclasses (isotypes), e.g., IgG1, IgG2, IgG3, IgG4, IgA, and IgA2. The heavy-chain constant domains (Fc) that correspond to the different classes of immunoglobulins are called α, δ, ε, γ, and μ, respectively. The subunit structures and three-dimensional configurations of different classes of immunoglobulins are well known.

The “light chains” of antibodies (immunoglobulins) from any vertebrate species can be assigned to one of two clearly distinct types, called kappa or (“κ”) and lambda or (“λ”), based on the amino acid sequences of their constant domains.

The terms “antigen-binding portion of an antibody,” “antigen-binding fragment,” “antigen-binding domain,” “antibody fragment” or a “functional fragment of an antibody” are used interchangeably in the present disclosure to mean one or more fragments of an antibody that retain the ability to specifically bind to an antigen (see, e.g., Holliger et al., Nature Biotech. 23 (9): 1126-1129 (2005)). Non-limiting examples of antibody fragments included within, but not limited to, the term “antigen-binding portion” of an antibody include (i) a Fab fragment, a monovalent fragment consisting of the V_{L}, V_{H}, C_{L} and C_{H1} domains; (ii) a F(ab′)₂ fragment, a bivalent fragment comprising two Fab fragments linked by a disulfide bridge at the hinge region; (iii) a Fd fragment consisting of the V_{H} and C_{H1} domains; (iv) a Fv fragment consisting of the V_{L} and V_{H} domains of a single arm of an antibody, (v) a dAb fragment (Ward et al., (1989) Nature 341:544-546), which consists of a V_{H} domain; and (vi) an isolated complementarity determining region (CDR). Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird et al. (1988) Science 242:423-426; and Huston et al. (1988) Proc. Natl. Acad. Sci. USA 85:5879-5883; and Osboum et al. (1998) Nat. Biotechnol. 16:778). Such single chain antibodies are also intended to be encompassed within the term “antigen-binding portion” of an antibody. Any V_{H} and V_{L} sequences of specific scFv can be linked to human immunoglobulin constant region cDNA or genomic sequences, in order to generate expression vectors encoding complete IgG molecules or other isotypes. V_{H} and V_{L} can also be used in the generation of Fab, Fv or other fragments of immunoglobulins using either protein chemistry or recombinant DNA technology. Other forms of single chain antibodies, such as diabodies are also encompassed.

“F(ab′)₂” and “Fab′” moieties can be produced by treating immunoglobulin (monoclonal antibody) with a protease such as pepsin and papain, and includes an antibody fragment generated by digesting immunoglobulin near the disulfide bonds existing between the hinge regions in each of the two H chains. For example, papain cleaves IgG upstream of the disulfide bonds existing between the hinge regions in each of the two H chains to generate two homologous antibody fragments in which an L chain composed of V_{L} (L chain variable region) and C_{L} (L chain constant region), and an H chain fragment composed of V_{H} (H chain variable region) and C_{Hγ1} (γ1 region in the constant region of H chain) are connected at their C terminal regions through a disulfide bond. Each of these two homologous antibody fragments is called Fab′. Pepsin cleaves IgG downstream of the disulfide bonds existing between the hinge regions in each of the two H chains to generate an antibody fragment slightly larger than the fragment in which the two above-mentioned Fab′ are connected at the hinge region. This antibody fragment is called F(ab′)₂.

The Fab fragment also contains the constant domain of the light chain and the first constant domain (C_{H}1) of the heavy chain. Fab′ fragments differ from Fab fragments by the addition of a few residues at the carboxyl terminus of the heavy chain C_{H}1 domain including one or more cysteine(s) from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

“Fv” is the minimum antibody fragment which contains a complete antigen-recognition and antigen-binding site. This region consists of a dimer of one heavy chain and one light chain variable domain in tight, non-covalent association. It is in this configuration that the three hypervariable regions of each variable domain interact to define an antigen-binding site on the surface of the V_{H}-V_{L} dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site.

“Single-chain Fv” or “sFv” antibody fragments comprise the V_{H} and V_{L} domains of an antibody, wherein these domains are present in a single polypeptide chain. In some embodiments, the Fv polypeptide further comprises a polypeptide linker between the V_{H} and V_{L} domains which enables the sFv to form the desired structure for antigen binding. For a review of sFv molecules, see, e.g., Pluckthun in The Pharmacology of Monoclonal Antibodies, Vol. 113, Rosenburg and Moore eds. Springer-Verlag, New York, pp. 269-315 (1994).

As used herein, “natural” or “naturally occurring” antibodies or antibody variable domains, refers to antibodies or antibody variable domains having a sequence of an antibody or antibody variable domain identified from a non-synthetic source, for example, from a germline sequence, or differentiated antigen-specific B cell obtained ex vivo, or its corresponding hybridoma cell line, or from the serum of an animal. These antibodies can include antibodies generated in any type of immune response, either natural or otherwise induced. Natural antibodies include the amino acid sequences, and the nucleotide sequences that constitute or encode these antibodies, for example, as identified in the Kabat database.

“Epitope” refers to that portion of an antigen or other macromolecule capable of forming a binding interaction that interacts with the variable region binding pocket of a binding protein. Such binding interaction can be manifested as an intermolecular contact with one or more amino acid residues of a CDR. Antigen binding can involve a CDR3 or a CDR3 pair. An epitope can be a linear peptide sequence (i.e., “continuous”) or can be composed of noncontiguous amino acid sequences (i.e., “conformational” or “discontinuous”). A binding protein can recognize one or more amino acid sequences; therefore an epitope can define more than one distinct amino acid sequence. Epitopes recognized by binding protein can be determined by peptide mapping and sequence analysis techniques well known to one of skill in the art. A “cryptic epitope” or a “cryptic binding site” is an epitope or binding site of a protein sequence that is not exposed or substantially protected from recognition within an unmodified polypeptide, or protein complex or multimer, but is capable of being recognized by a binding protein to the proteolyzed polypeptide, or non complexed, dissociated polypeptide. Amino acid sequences that are not exposed, or are only partially exposed, in the unmodified, multimeric polypeptide structure are potential cryptic epitopes. If an epitope is not exposed, or only partially exposed, then it is likely that it is buried within the interior of the polypeptide, or masked in the polypeptide complex by the binding of other proteins or factors. Candidate cryptic epitopes can be identified, for example, by examining the three-dimensional structure of an unmodified polypeptide.

The term “specific” is applicable to a situation in which one member of a specific binding pair will not show any significant binding to molecules other than its specific binding partner(s). The term is also applicable where e.g., an antigen binding domain is specific for a particular epitope which is carried by a number of antigens, in which case the specific binding member carrying the antigen binding domain will be able to bind to the various antigens carrying the epitope.

The term “binding” refers to a direct association between two molecules, due to, for example, covalent, electrostatic, hydrophobic, and ionic and/or hydrogen-bond interactions, including interactions such as salt bridges and water bridges.

The phrase “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz, G. E. and R. H. Schirmer, Principles of Protein Structure, Springer-Verlag). According to such analyses, groups of amino acids can be defined where amino acids within a group exchange preferentially with each other, and therefore resemble each other most in their impact on the overall protein structure (Schulz, G. E. and R. H. Schirmer, Principles of Protein Structure, Springer-Verlag).

Examples of amino acid groups defined in this manner include: a “charged/polar group,” consisting of Glu, Asp, Asn, Gln, Lys, Arg and His; an “aromatic, or cyclic group,” consisting of Pro, Phe, Tyr and Trp; and an “aliphatic group” consisting of Gly, Ala, Val, Leu, lie, Met, Ser, Thr and Cys.

Within each group, subgroups can also be identified, for example, the group of charged/polar amino acids can be sub-divided into the sub-groups consisting of the “positively-charged sub-group,” consisting of Lys, Arg and His; the negatively-charged sub-group,” consisting of Glu and Asp, and the “polar sub-group” consisting of Asn and Gln.

The aromatic or cyclic group can be sub-divided into the sub-groups consisting of the “nitrogen ring sub-group,” consisting of Pro, His and Trp; and the “phenyl sub-group” consisting of Phe and Tyr.

The aliphatic group can be sub-divided into the sub-groups consisting of the “large aliphatic non-polar sub-group,” consisting of Val, Leu and lie; the “aliphatic slightly-polar sub-group,” consisting of Met, Ser, Thr and Cys; and the “small-residue sub-group,” consisting of Gly and Ala.

Examples of conservative mutations include amino acid substitutions of amino acids within the sub-groups above, for example, Lys for Arg and vice versa such that a positive charge can be maintained; Glu for Asp and vice versa such that a negative charge can be maintained; Ser for Thr such that a free —OH can be maintained; and Gln for Asn such that a free —NH₂ can be maintained.

“Semi-conservative mutations” include amino acid substitutions of amino acids with the same groups listed above, that do not share the same sub-group. For example, the mutation of Asp for Asn or Asn for Lys all involve amino acids within the same group, but different sub-groups.

“Non-conservative mutations” involve amino acid substitutions between different groups, for example Lys for Leu, or Phe for Ser etc. The term “Non conservative amino mutation: also refers to the replacement of any amino acid with a non-naturally occurring, synthetically produced amino acid derivative.

The terms “cells,” “cell cultures,” “cell line,” “recombinant host cells,” “recipient cells,” and “host cells” are often used interchangeably and will be clear from the context in which they are used. These terms include the primary subject cells and any progeny thereof, without regard to the number of transfers. It should be understood that not all progeny are exactly identical to the parental cell (due to deliberate or inadvertent mutations or differences in environment), however, such altered progeny are included in these terms, so long as the progeny retain the same functionality as that of the originally transformed cell. For example, though not limited to, such a characteristic might be the ability to produce a particular recombinant protein.

The term “immortalized” refers in general to the cell cultures and cell lines obtained after exposing the selected and stimulated population of cells to a viral immortalizing agent. Typically, immortalized cells exhibit sustained cell growth or at least for a period of time and/or for a number of cell divisions largely superior (i.e., at least 3-fold better, more preferably at least 5 times better, or even more preferably at least about 10-fold better) than non transformed primary cells. Cells are defined as “immortalized” when they show, continuous growth and proliferation when grown in optimized cell culture conditions or a period of at least three months, or more preferably about six months in continuous culture.

The term “immortalized antibody-secreting cells” refers to antibody-secreting cells that, following exposure to a viral immortalizing agent, show continuous growth, proliferation, and maintain the secretion of antibodies when grown in optimized cell culture conditions.

The term “viral immortalizing agent” refers to any kind of viral particle, DNA, or protein, which allows generating immortalized cells from primary cells isolated from biological samples. In the present case, the primary cells are antibody-secreting cells, in particular human B cells, for which different viral immortalizing agents have been identified.

The term “polyclonal activator” means a molecule or compound or a combination thereof that activates B lymphocytes irrespective of their antigenic specificity. A range of different molecules may be used as the polyclonal activator any of the methods of the disclosure. Preferred polyclonal activators include immunostimulatory nucleic acid molecules, for example as disclosed in PCT publication WOV9818810A1, entitled IMMUNOSTIMULATORY NUCLEIC ACID MOLECULES” The term “immunostimulatory nucleic acid molecules”, refers to a nucleic acid molecule, which contains an unmethylated cytosine, guanine dinucleotide sequence (i.e., “CpG DNA” or DNA containing a cytosine followed by guanosine and linked by a phosphate bond) and stimulates (e.g., has a mitogenic effect on, or induces or increases cytokine expression by) a vertebrate lymphocyte. An immunostimulatory nucleic acid molecule can be double-stranded or single-stranded. Generally, double-stranded molecules are more stable in vivo, while single-stranded molecules have increased immune activity. In a preferred embodiment, the immunostimulatory nucleic acid contains a consensus mitogenic CpG motif represented by the formula:

5′ X1CGX2 3′ wherein X₁ is selected from the group consisting of A, G and T; and X₂ is C or T.

In a particularly preferred embodiment, immunostimulatory nucleic acid molecules are between 2 to 100 base pairs in size and contain a consensus mitogenic CpG motif represented by the formula:

5′ X1X2CGX3X4 3′ wherein C and G are unmethylated, X₁, X₂, X₃ and X₄ are nucleotides.

“Homology” or “identity” or “similarity” refers to sequence similarity between two peptides or between two nucleic acid molecules. Homology and identity can each be determined by comparing a position in each sequence which can be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology/similarity or identity refers to a function of the number of identical or similar amino acids at positions shared by the compared sequences. A sequence which is “unrelated” or “non-homologous” shares less than 40% identity, less than 35% identity, less than 30% identity, or less than 25% identity with a sequence of the present disclosure. In comparing two sequences, the absence of residues (amino acids or nucleic acids) or presence of extra residues also decreases the identity and homology/similarity.

The term “homology” describes a mathematically based comparison of sequence similarities which is used to identify genes or proteins with similar functions or motifs. The nucleic acid and protein sequences of the present disclosure can be used as a “query sequence” to perform a search against public databases to, for example, identify other family members, related sequences or homologs. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to nucleic acid molecules of the disclosure. BLAST protein searches can be performed with the XBLAST program, score=50, wordlength=3 to obtain amino acid sequences homologous to protein molecules of the disclosure. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and BLAST) can be used (See ncbi.nlm.nih.gov).

As used herein, “identity” means the percentage of identical nucleotide or amino acid residues at corresponding positions in two or more sequences when the sequences are aligned to maximize sequence matching, i.e., taking into account gaps and insertions. Identity can be readily calculated by known methods, including but not limited to those described in (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48: 1073 (1988). Methods to determine identity are designed to give the largest match between the sequences tested. Moreover, methods to determine identity are codified in publicly available computer programs. Computer program methods to determine identity between two sequences include, but are not limited to, the GCG program package (Devereux, J., et al., Nucleic Acids Research 12(1): 387 (1984)), BLASTP, BLASTN, and FASTA (Altschul, S. F. et al., J. Molec. Biol. 215: 403-410 (1990) and Altschul et al. Nuc. Acids Res. 25: 3389-3402 (1997)). The BLAST X program is publicly available from NCBI and other sources (BLAST Manual, Altschul, S., et al., NCBI NLM NIH Bethesda, Md. 20894; Altschul, S., et al., J. Mol. Biol. 215: 403-410 (1990). The well known Smith Waterman algorithm can also be used to determine identity.

A “heterologous” region of a DNA sequence is an identifiable segment of DNA within a larger DNA sequence that is not found in association with the larger sequence in nature. Thus, when the heterologous region encodes a mammalian gene, the gene can usually be flanked by DNA that does not flank the mammalian genomic DNA in the genome of the source organism. Another example of a heterologous coding sequence is a sequence where the coding sequence itself is not found in nature (e.g., a cDNA where the genomic coding sequence contains introns or synthetic sequences having codons or motifs different than the unmodified gene). Allelic variations or naturally-occurring mutational events do not give rise to a heterologous region of DNA as defined herein.

As used herein, the term “isolated” means that the referenced material is removed from the environment in which it is found. Thus, an isolated biological material can be free of cellular components, i.e., components of the cells in which the material is found or produced. In the case of nucleic acid molecules, an isolated nucleic acid includes a PCR product, an isolated mRNA, a cDNA, or a restriction fragment. Isolated nucleic acid molecules include sequences inserted into plasmids, cosmids, artificial chromosomes, and the like. Thus, the term “isolated nucleic acid” includes a recombinant nucleic acid. An isolated protein may be associated with other proteins or nucleic acids, or both, with which it associates in the cell, or with cellular membranes if it is a membrane-associated protein. An isolated material may be, but need not be, purified.

The term “purified” as used herein refers to material that has been isolated under conditions that reduce or eliminate the presence of unrelated materials, i.e., contaminants, including native materials from which the material is obtained. For example, a purified protein is preferably substantially free of other proteins or nucleic acids with which it is associated in a cell. Methods for purification are well-known in the art, and examples are discussed below. As used herein, the term “substantially free” is used operationally, in the context of analytical testing of the material. Preferably, purified material substantially free of contaminants is at least 50% pure; more preferably, at least 90% pure, and more preferably still at least 99% pure. Purity can be evaluated by chromatography, gel electrophoresis, immunoassay, composition analysis, biological assay, and other methods known in the art

A purified material may contain less than about 50%, preferably less than about 75%, and most preferably less than about 90%, of the cellular components with which it was originally associated. The term “substantially pure” indicates the highest degree of purity, which can be achieved using conventional purification techniques known in the art.

A “sample” as used herein refers to a biological material which can be tested, e.g., for the presence of osteopontin polypeptides or anti-osteopontin antibodies. Such samples can be obtained from any source, including tissue, tumorigenic tissue; blood and blood cells, including circulating hematopoietic stem cells (for possible detection of protein or nucleic acids), plural effusions, cerebrospinal fluid (CSF), ascites fluid, and cell culture.

The term “subject” in the context of the present disclosure is preferably a mammal. The mammal can be a human, non human primate, mouse, rat, rabbit, cat, horse or cow, but is not limited to these examples. Mammals other than humans can be advantageously used in place of human donors that represent animal models of the corresponding human disease, such as an osteopontin related disease. A subject may be male or female. A subject may be a human patient that has been diagnosed with osteoarthritis, rheumatoid arthritis, multiple sclerosis, or any other osteopontin related disease.

The term “molecule” means any distinct or distinguishable structural unit of matter comprising one or more atoms, and includes, for example, polypeptides and polynucleotides.

An “immunogenic composition” of the disclosure, as used herein, refers to any composition that elicits an immune response in an animal. An “immune response” is the reaction of the body to foreign substances, without implying a physiologic or pathologic consequence of such a reaction, i.e., without necessarily conferring protective immunity on the animal. An immune response may include one or more of the following: (a) a cell mediated immune response, which involves the production of lymphocytes by the thymus (T cells) in response to exposure to the antigen; and/or (b) a humoral immune response, which involves production of plasma lymphocytes (B cells) in response to antigen exposure with subsequent antibody production.

The term “vaccine”, as used herein, broadly refers to any compositions that may be administered to an animal to illicit a protective immune response to the vaccine or co-administered antigen. The terms “protect”, “protective “immune response” or “protective immunity”, as used herein describes the development of antibodies or cellular systems that specifically recognize the vaccine antigen.

The term “effective amount” refers to an amount of a compound or compositions that is sufficient to provide a desired result. Thus, as used to describe a vaccine, an effective amount refers to an amount of a compound or composition (e.g., an antigen) that is sufficient to produce or elicit a protective immune response. An effective amount with respect to an immunological composition is an amount that is sufficient to elicit an immune response, whether or not the response is protective.

In accordance with the present disclosure, there may be employed conventional molecular biology, microbiology and recombinant DNA techniques within the skill of the art. Such techniques are explained fully in the literature. See, e.g., Sambrook, Fitsch & Maniatis, Molecular Cloning: A Laboratory Manual, Second Edition Cold Spring Harbor Laboratory Press, Cold Spring Harbor, N.Y. (1989) (referred to herein as “Sambrook et al., 1989”).

The term “polynucleotide” or “nucleic acid” sequence as used herein refers to and means any chain of two or more nucleotides. Nucleotides are phosphate esters of pentoses in which a nitrogenous base is linked to C(1) of the sugar residue. Such bases are typically adenine, guanine, cytosine, uracil, thymine, and hypoxanthine, but some may be modified bases, for example, thio-uracil, thio-guanine and fluoro-uracil.

A nucleotide sequence frequently carries genetic information, including the information used by cellular machinery to make proteins and enzymes. The terms include genomic DNA, cDNA, RNA, any synthetic and genetically manipulated polynucleotides, and both sense and antisense polynucleotides. This includes single- and double-stranded molecules as well as backbone modifications thereof (for example, methylphosphonate linkages); i.e., DNA-DNA, DNA-RNA, and RNA-RNA hybrids as well as “protein nucleic acids” (PNA) formed by conjugating bases to an amino acid backbone.

The polynucleotides herein may be flanked by natural regulatory sequences, or may be associated with heterologous sequences, including promoters, enhancers, response elements, signal sequences, polyadenylation sequences, introns, 5′- and 3′-non-coding regions and the like. The nucleic acids may also be modified by many means known in the art. Non-limiting examples of such modifications include methylation, “caps”, substitution of one or more of the naturally occurring nucleotides with an analog, and internucleotide modifications. Polynucleotides may contain one or more additional covalently linked moieties, such as proteins (e.g., nucleases, toxins, antibodies, signal peptides, poly-L-lysine, etc.), intercalators (e.g., acridine, psoralen, etc.), chelators (e.g., metals, radioactive metals, iron, oxidative metals, etc.) and alkylators to name a few. The polynucleotides may be derivatized by formation of a methyl or ethyl phosphotriester or an alkyl phosphoramidite linkage. Furthermore, the polynucleotides herein may also be modified with a label capable of providing a detectable signal, either directly or indirectly. Exemplary labels include radioisotopes, fluorescent molecules, biotin and the like. Other non-limiting examples of modification which may be made are provided, below, in the description of the present disclosure.

A “polypeptide” is a chain of chemical building blocks called amino acids that are linked together by chemical bonds called “peptide bonds”. The term “protein” refers to polypeptides that contain the amino acid residues encoded by a gene or by a nucleic acid molecule (e.g., an mRNA or a cDNA) transcribed from that gene either directly or indirectly. Optionally, a protein may lack certain amino acid residues that are encoded by a gene or by an mRNA. For example, a gene or mRNA molecule may encode a sequence of amino acid residues on the N-terminus of a protein (i.e., a signal sequence) that is cleaved from, and therefore may not be part of, the final protein. A protein or polypeptide, including an enzyme, may be a “native” or “wild-type”, meaning that it occurs in nature; or it may be a “mutant”, a “variant,” or may be referred to as “modified”, meaning that it has been made, altered, derived, or is in some way different or changed from a native protein or from another mutant.

A “ligand” is, broadly speaking, any molecule that binds to another molecule. In preferred embodiments, the ligand is either a soluble molecule or the smaller of the two molecules or both. The other molecule may be referred to as a “receptor”. In preferred embodiments, both a ligand and its receptor are molecules (preferably proteins or polypeptides) produced by cells. In one aspect, a ligand is a soluble molecule and the receptor is an integral membrane protein (i.e., a protein expressed on the surface of a cell).

“Amplification” of a polynucleotide, as used herein, denotes the use of polymerase chain reaction (PCR) to increase the concentration of a particular DNA sequence within a mixture of DNA sequences. For a description of PCR see Saiki et al., Science, 239: 487 (1988).

“Chemical sequencing” of DNA denotes methods such of Maxam-Gilbert (see Maxam & Gilbert, Proc. Natl. Acad. Sci. U.S.A. (1977), 74: 560), in which DNA is cleaved using individual base-specific reactions.

“Enzymatic sequencing” of DNA denotes methods such as that of Sanger (Sanger et al., Proc. Natl. Acad. Sci. U.S.A. (1977), 74: 5463) and variations thereof well known in the art, in a single-stranded DNA is copied and randomly terminated using DNA polymerase.

A “gene” is a sequence of nucleotides which code for a functional “gene product”. Generally, a gene product is a functional protein. However, a gene product can also be another type of molecule in a cell, such as RNA (e.g., a tRNA or an rRNA). For the purposes of the present disclosure, a gene product also refers to an mRNA sequence which may be found in a cell. For example, measuring gene expression levels according to the disclosure may correspond to measuring mRNA levels. A gene may also comprise regulatory (i.e., non-coding) sequences as well as coding sequences. Exemplary regulatory sequences include promoter sequences, which determine, for example, the conditions under which the gene is expressed. The transcribed region of the gene may also include untranslated regions including introns, a 5′-untranslated region (5′-UTR) and a 3′-untranslated region (3′-UTR).

A “coding sequence” or a sequence “encoding” an expression product, such as a RNA, polypeptide, protein or enzyme, is a nucleotide sequence that, when expressed, results in the production of that RNA, polypeptide, protein or enzyme; i.e., the nucleotide sequence “encodes” that RNA or it encodes the amino acid sequence for that polypeptide, protein or enzyme.

An “expression control sequence” is a DNA regulatory region capable of facilitating the information in a gene or DNA sequence to become manifest, e.g., producing RNA (rRNA or mRNA) or a protein by activating the cellular functions involved in transcription and translation of a corresponding gene or DNA sequence. For example, an expression control sequence may include a promoter sequence, which is a DNA regulatory region capable of binding RNA polymerase in a cell and initiating transcription of a downstream (3′ direction) coding sequence. The expression control sequence may also include an enhancer sequence which is a DNA sequence capable of increasing the transcription of a gene into mRNA. The constructs of the present disclosure may contain a promoter alone or in combination with an enhancer, and these elements need not be contiguous.

A coding sequence is “under the control of” or is “operatively associated with” transcriptional and translational control sequences in a cell when RNA polymerase transcribes the coding sequence into RNA, which is then trans-RNA spliced (if it contains introns) and, if the sequence encodes a protein, is translated into that protein.

The term “transfection” means the introduction of a foreign nucleic acid into a cell. The term “transformation” means the introduction of a “foreign” (i.e., extrinsic or extracellular) gene, DNA or RNA sequence into a host cell so that the host cell will express the introduced gene or sequence to produce a desired substance, in this disclosure typically an RNA coded by the introduced gene or sequence, but also a protein or an enzyme coded by the introduced gene or sequence. The introduced gene or sequence may also be called a “cloned” or “foreign” gene or sequence, may include regulatory or control sequences (e.g., start, stop, promoter, signal, secretion or other sequences used by a cell's genetic machinery). The gene or sequence may include nonfunctional sequences or sequences with no known function. A host cell that receives and expresses introduced DNA or RNA has been “transformed”. The DNA or RNA introduced to a host cell can come from any source, including cells of the same genus or species as the host cell or cells of a different genus or species.

The terms “vector”, “cloning vector” and “expression vector” mean the vehicle by which a DNA or RNA sequence (e.g., a foreign gene) can be introduced into a host cell so as to transform the host and promote expression (e.g., transcription and translation) of the introduced sequence. Vectors may include plasmids, phages, viruses, etc. and are discussed in greater detail below.

The term “expression system” means a host cell and compatible vector under suitable conditions, e.g., for the expression of a protein coded for by foreign DNA carried by the vector and introduced to the host cell. Common expression systems include E. coli host cells and plasmid vectors, insect host cells such as Sf9, Hi5 or S2 cells and Baculovirus vectors, Drosophila cells (Schneider cells) and expression systems, and mammalian host cells and vectors.

The term “heterologous” refers to a combination of elements not naturally occurring. For example, heterologous DNA refers to DNA that is not naturally located in the cell, or in a chromosomal site of the cell. Preferably, heterologous DNA includes a gene foreign to the cell. A heterologous expression regulatory element is a regulatory element operatively associated with a different gene that the one it is operatively associated with in nature.

The term “homologous” refers to the relationship between two proteins that possess a “common evolutionary origin”, including proteins from superfamilies (e.g., the immunoglobulin superfamily) in the same species of animal, as well as homologous proteins from different species of animal (for example, myosin light chain polypeptide, etc.; see Reeck et al., Cell, 50:667, 1987). Such proteins (and their encoding nucleic acids) have sequence homology, as reflected by their sequence similarity, whether in terms of percent identity or by the presence of specific residues or motifs and conserved positions.

The term “sequence similarity” refers to the degree of identity or correspondence between nucleic acid or amino acid sequences that may or may not share a common evolutionary origin (see Reeck et al., supra). However, in common usage and in the instant application, the term “homologous”, when modified with an adverb such as “highly”, may refer to sequence similarity and may or may not relate to a common evolutionary origin.

In specific embodiments, two nucleic acid sequences are “substantially homologous” or “substantially similar” when at least about 85%, and more preferably at least about 90% or at least about 95% of the nucleotides match over a defined length of the nucleic acid sequences, as determined by a sequence comparison algorithm known such as BLAST, FASTA, DNA Strider, CLUSTAL, etc. An example of such a sequence is an allelic or species variant of the specific genes of the present disclosure. Sequences that are substantially homologous may also be identified by hybridization, e.g., in a Southern hybridization experiment under, e.g., stringent conditions as defined for that particular system.

Similarly, in particular embodiments of the disclosure, two amino acid sequences are “substantially homologous” or “substantially similar” when greater than 80% of the amino acid residues are identical. Two sequences are considered “functionally identical” when greater than about 90% of the amino acid residues are identical. (i.e.). Preferably the similar or homologous polypeptide sequences are identified by alignment using, for example, the GCG (Genetics Computer Group, Version 7, Madison, Ws.) pileup program, or using any of the programs and algorithms described above.

The terms “mutant” and “mutation” mean any detectable change in genetic material, e.g., DNA, or any process, mechanism or result of such a change. This includes gene mutations, in which the structure (e.g., DNA sequence) of a gene is altered, any gene or DNA arising from any mutation process, and any expression product (e.g., RNA, protein or enzyme) expressed by a modified gene or DNA sequence. The term “variant” may also be used to indicate a modified or altered gene, DNA sequence, RNA, enzyme, cell, etc. For example, the present disclosure relates to altered or “chimeric” RNA molecules that comprise an rRNA sequence that is altered by inserting a heterologous RNA sequence that is not naturally part of that sequence or is not naturally located at the position of that rRNA sequence. Such chimeric RNA sequences, as well as DNA and genes that encode them, are also referred to herein as “mutant” sequences. “Sequence-conservative variants” of a polynucleotide sequence are those in which a change of one or more nucleotides in a given codon position results in no alteration in the amino acid encoded at that position.

As used herein, the term “oligonucleotide” refers to a nucleic acid, generally at least 10, preferably at least 15, and more preferably at least 20 nucleotides, preferably no more than 100 nucleotides.

Oligonucleotides can be labeled, e.g., ³²P-nucleotides, or nucleotides to which a label, such as biotin or a fluorescent dye (for example, Cy3 or Cy5) has been covalently conjugated. In one embodiment, a labeled oligonucleotide can be used as a probe to detect the presence of a nucleic acid. In another embodiment, oligonucleotides (one or both of which may be labeled) can be used as PCR primers, either for cloning full length or a fragment of Osteopontin, or to detect the presence of nucleic acids encoding Osteopontin. In a further embodiment, an oligonucleotide of the disclosure can form a triple helix with an Osteopontin DNA molecule. Generally, oligonucleotides are prepared synthetically, preferably on a nucleic acid synthesizer. Accordingly, oligonucleotides can be prepared with non-naturally occurring phosphoester analog bonds, such as thioester bonds, etc.

A sequence that is “complementary” to a portion of a nucleic acid refers to a sequence having sufficient complementarity to be able to hybridize with the nucleic acid and form a stable duplex. The ability of nucleic acids to hybridize will depend both on the degree of sequence complementarity and the length of the antisense nucleic acid. Generally, however, the longer the hybridizing nucleic acid, the more base mismatches it may contain and still forms a stable duplex (or triplex in triple helix methods). A tolerable degree of mismatch can be readily ascertained, etc., by using standard procedures to determine the melting temperature of a hybridized complex.

As used herein, the terms “osteopontin” or “osteopontin peptide,” refer to a form of osteopontin or a fragment thereof, capable of performing its intended function in vivo, or in vitro. Examples of osteopontin peptides useful in the disclosure include, but are not limited to recombinant full length osteopontin, e.g., a human or murine recombinant osteopontin, and all naturally occurring forms of osteopontin, including phosphorylated, glycosylated and proteolytic derivatives of osteopontin formed in situ. A full length amino acid sequence of human osteopontin, according to Kiefer M C et al. (1989) Nucleic Acids Research 17(8): 3306, is shown below.

(SEQ ID NO: 1)
MRIAVICFCLLGITCAIPVKQADSGSSEEKQLYNKYPDAVATWLNPDPSQ
KQNLLAPQNAVSSEETNDFKQETLPSKSNESHDHMDDMDDEDDDDHVDSQ
DSIDSNDSDDVDDTDDSHQSDESHHSDESDELVTDFPTDLPATEVFTPVV
PTVDTYDGRGDSVVYGLRSKSKKFRRPDIQYPDATDEDITSHMESEELNG
AYKAIPVAQDLNAPSDWDSRGKDSYETSQLDDQSAETHSHKQSRLYKRKA
NDESNEHSDVIDSQELSKVSREFHSHEFHSHEDMLVVDPKSKEEDKHLKF
RISHELDSASSEVN

Additional forms of osteopontin of relevance to the disclosure include Osteopontin-R, osteopontin L, and osteopontin-166 as defined below:

Osteopontin-R
(SEQ ID NO: 2)
MRIAVICFCLLGITCAIPVKQADSGSSEEKQLYNKYPDAVATWLNPDPSQ
KQNLLAPQNAVSSEETNDFKQETLPSKSNESHDHMDDMDDEDDDDHVDSQ
DSIDSNDSDDVDDTDDSHQSDESHHSDESDELVTDFPTDLPATEVFTPVV
PTVDTYDGRGDSVVYGLR
Osteopontin-L
(SEQ ID NO: 3)
MRIAVICFCLLGITCAIPVKQADSGSSEEKQLYNKYPDAVATWLNPDPSQ
KQNLLAPQNAVSSEETNDFKQETLPSKSNESHDHMDDMDDEDDDDHVDSQ
DSIDSNDSDDVDDTDDSHQSDESHHSDESDELVTDFPTDLPATEVFTPVV
PTVDTYDGRGDSVVYGL
Osteopontin-166
(SEQ ID NO: 4)
MRIAVICFCLLGITCAIPVKQADSGSSEEKQLYNKYPDAVATWLNPDPSQ
KQNLLAPQNAVSSEETNDFKQETLPSKSNESHDHMDDMDDEDDDDHVDSQ
DSIDSNDSDDVDDTDDSHQSDESHHSDESDELVTDFPTDLPATEVFTPVV
PTVDTYDGRGDSVVYG

The term “calcification” refers to a process whereby a tissue or non-cellular material in the body, becomes hardened as a result of the deposition of insoluble salts of calcium, such as calcium phosphate or calcium carbonate, or sometimes insoluble salts of magnesium.

The term “anti-calcification activity” refers to the inhibition, prevention or amelioration of the ectopic calcification of tissues in response to an injury, pathology, or condition, or the inhibition, prevention or amelioration of ectopic calcification on or around an implanted prosthetic device. “Anti-calcification activity” includes preventing calcification from occurring in an individual who may be predisposed to developing ectopic calcification, but who does not yet experience or exhibit symptoms of ectopic calcification (prophylactic treatment), or inhibiting ectopic calcification (slowing or arresting further deposition of calcium salts) and/or causing regression or resorption of the calcified deposits.

“Osteopontin related disease” includes, multiple sclerosis (MS), atherosclerosis and related coronary diseases, rheumatoid arthritis, lupus, nephritis, cerebritis, Crohn's disease, osteoporosis, inflammatory bowel disorder, breast cancer, ovarian cancer, pancreatic cancer, bladder cancer, lung cancer, colon cancer, gastric carcinomas, esophageal carcinomas, squamous cell carcinomas of the head or neck, prostate cancer, thyroid cancer, melanoma, kidney cancers, renal cell carcinomas, endometrial cancer, small intestine cancer, duodenal cancer, cholangiocarcinoma, astrocytoma, AIDS lymphoma, follicular lymphoma, T-cell lymphoma, B-cell lymphoma, proliferative retinopathy, vitreoretinopathy, diabetic retinopathy, macular degeneration, non-HIV dementia, HIV- and AIDS-associated dementia, focal segmental glomerulosclerosis, membrane proliferative glomerulonephropathy, psoriasis, herpes virus associated disease, Castleman's disease, Kaposi's sarcoma, Alzheimer's disease, type 2 diabetes, cardiac fibrosis and angiotensin type II associated hypertension, mast cell produced IgE mediated hypersensitivity immune reactions, lupus, vasculitis, prelymphomatic or lymphoproliferation related autoimmune conditions. The term “Osteopontin related disease” also includes animal models of the corresponding human conditions

### Methods for Obtaining Novel Human Auto-Antibodies

In one aspect, the present disclosure includes a method for selecting a therapeutic antibody to osteopontin comprising the steps of; screening a subject with osteoarthritis, multiple sclerosis or rheumatoid arthritis, for osteopontin specific antibodies, and isolating or amplifying one or more nucleic acids from the subject's B cells encoding an antibody heavy or light chain, or portion thereof, that binds to osteopontin.

In one aspect, of this method the one or more nucleic acids obtained from the subject's B cells are obtained from the isolated B cells without B-cell immortalization. In another aspect of this method, the B cells are immortalized.

In another aspect, the present disclosure includes a method for selecting a human therapeutic antibody to osteopontin comprising the steps of;

a) isolating antibody-secreting cells obtained from a human donor with osteoarthritis, multiple sclerosis or rheumatoid arthritis;

b) immortalizing the cells isolated in step a) to produce immortalized antibody-secreting cell cultures;

c) screening the immortalized antibody-secreting cell cultures obtained in step b) for cultures producing osteopontin specific antibodies;

d) isolating or amplifying one or more nucleic acids from the immortalized antibody-secreting cell cultures producing osteopontin specific antibodies of step c) encoding an antibody heavy or light chain, or portion thereof, that binds to osteopontin.

In another embodiment, the present disclosure includes a method for selecting a human therapeutic antibody for treating an osteopontin related disease, comprising the steps of;

a) isolating antibody-secreting cells obtained from a human donor with osteoarthritis multiple sclerosis or rheumatoid arthritis;

b) immortalizing the cells isolated in step a) to produce immortalized antibody-secreting cell cultures;

c) screening the immortalized antibody-secreting cell cultures obtained in step b) for cultures producing osteopontin specific antibodies;

d) isolating or amplifying one or more nucleic acids from the immortalized antibody-secreting cell cultures producing osteopontin specific antibodies of step c) encoding an antibody heavy or light chain, or portion thereof, that binds to osteopontin.

In another aspect, the present disclosure includes a method for selecting a human therapeutic antibody for treating multiple sclerosis comprising the steps of;

a) isolating antibody-secreting cells obtained from a human donor with osteoarthritis, multiple sclerosis or rheumatoid arthritis;

b) immortalizing the cells isolated in step a) to produce immortalized antibody-secreting cell cultures;

c) screening the immortalized antibody-secreting cell cultures obtained in step b) for cultures producing osteopontin specific antibodies;

d) isolating or amplifying one or more nucleic acids from the immortalized antibody-secreting cell cultures producing osteopontin specific antibodies of step c) encoding an antibody heavy or light chain, or portion thereof, that binds to osteopontin.

In another embodiment, the present disclosure includes a method for selecting a human therapeutic antibody for treating rheumatoid arthritis, comprising the steps of;

a) isolating immortalized antibody-secreting cells obtained from a human donor with osteoarthritis, multiple sclerosis or rheumatoid arthritis;

b) immortalizing the cells isolated in step a) to produce immortalized antibody-secreting cell cultures;

c) screening the immortalized antibody-secreting cell cultures obtained in step b) for cultures producing osteopontin specific antibodies;

d) isolating or amplifying one or more nucleic acids from the immortalized antibody-secreting cell cultures producing osteopontin specific antibodies of step c) encoding an antibody heavy or light chain, or portion thereof, that binds to osteopontin.

In another embodiment, the present disclosure includes a method for selecting a human therapeutic antibody for treating osteoarthritis comprising the steps of;

a) isolating antibody-secreting cells obtained from a human donor with osteoarthritis, multiple sclerosis or rheumatoid arthritis;

b) immortalizing the cells isolated in step a) to produce immortalized antibody-secreting cell cultures;

c) screening the immortalized antibody-secreting B cell cultures obtained in step b) for cultures producing osteopontin specific antibodies;

d) isolating or amplifying one or more nucleic acids from the immortalized antibody-secreting cell cultures producing osteopontin specific antibodies of step c) encoding an antibody heavy or light chain, or portion thereof, that binds to osteopontin.

In yet another embodiment, the present disclosure includes a process for preparing immortalized antibody-secreting cell clones expressing a therapeutic antibody to Osteopontin, to treat an osteopontin related disease comprising the steps of;

a) isolating antibody-secreting cells obtained from a human donor with osteoarthritis, multiple sclerosis or rheumatoid arthritis;

b) immortalizing the antibody-secreting cells by transforming antibody-secreting cells with Epstein Barr virus (EBV) in the presence of a polyclonal B cell activator;

c) screening the immortalized antibody-secreting cells for the expression of antibodies that bind to osteopontin, and

d) selecting immortalized antibody-secreting cells that exhibit antibodies that specifically bind to osteopontin.

In one aspect, of any of the claimed methods, screening may include subtractive immunopurification or immunodeletion approaches to selectively remove antibodies, and or identify antibodies to osteopontin that occur in one patient group compared to another patient group, or to a normal group. Representative antibody subtractive approaches include for example those listed in US20060154302A1 entitled “Iterative, subtractive immunoaffinity method for proteome analyte enrichment” WO06068646A1 entitled “METHODS FOR THE IDENTIFICATION AND THE ISOLATION OF EPITOPE SPECIFIC ANTIBODIES” and WO9830910A1 entitled “SUBTRACTIVE ANTIBODY SCREENING (SAS) AND USES THEREFOR”

In another aspect of any of these methods, the antibodies of the disclosure are obtained from a blood bank containing blood pooled from a number of human donors which has been matched for stage of disease progression, and current disease status such as active disease (“flare up”) or remission.

In another aspect, the antibodies of the disclosure are obtained from a blood bank containing blood pooled from a number of non-human subjects which has been matched for stage of disease progression, and current disease status such as active disease (“flare up”), or remission.

### Patient Profiling

In one aspect, of the claimed methods, the antibodies of the Disclosure are obtained from human B cells derived from pre-selected donors who have been diagnosed with osteoarthritis (OA). In one embodiment the donor has been diagnosed with early to middle stage of osteoarthritis. In one aspect, of this embodiment, the donor has osteoarthritis of the knee. In one aspect, the donor is in stable remission, meaning that one or more of the patient's symptoms of osteoarthritis have progressively decreased, or have remained substantially unchanged for at least about three months, about six months, or about twelve months or longer.

Osteoarthritis is a degenerative disease of the joints characterized by degradation of the hyaline articular cartilage and remodeling of the subchondral bone with sclerosis. Clinical problems include pain and joint stiffness often leading to significant disability and joint replacement.

Osteoarthritis exhibits a clear predilection for specific joints; it appears most commonly in the hip and knee joints and lumbar and cervical spine, as well as in the distal interphalangeal and the first carpometacarpal (base of thumb) and proximal interphalangeal joints of the hand; however, patients with osteoarthritis may have 1, a few, or all of these sites affected.

According to a conservative estimate, greater than 70% of the population of the United States at age 65 years is affected by the disease, reflecting its age dependence. Epidemiological and genetic studies have shown that osteoarthritis is a polygenic disease. Susceptibility to osteoarthritis of the hip, and female specific susceptibility to osteoarthritis is associated with variation in the frizzled-related protein (Loughlin, J. et al., Proc. Nat. Acad. Sci. 101: 9757-9762, 2004). Other forms, of osteoarthritis such as osteoarthritis of the hand and osteoarthritis of the knee/hip are associated with variation in the matrilin-3 and asporin genes, respectively (Stefansson, et al., Am. J. Hum. Genet. 72: 1448-1459, 2003, Kizawa, et al., Nature Genet. 37: 138-144, 2005). A locus for generalized osteoarthritis has been mapped to chromosome 2q33.3, and variation in the growth differentiation factor 5 gene is associated with osteoarthritis of the hip (Miyamoto et al., (2007) Nat Genet. 39(4) 529-33). Susceptibility to osteoarthritis of the knee has been mapped to chromosome 3p24.3 (Miyamoto, et al. Nature Genet. 40: 994-998, 2008).

The symptoms of OA usually begin after age 40 and can vary considerably from one person to another. Consistent with the diversity of symptoms auto-antibodies to osteopontin may be targeted to a range of function directing regions of the protein and therefore impact disease progression via distinct pathways, ultimately resulting in an acceleration of disease progression, or in some cases actually inhibiting or reversing disease progression Typically symptoms of OA include the following:

Pain—The main symptom of OA is joint pain that is worse with activity and relieved by rest. In severe cases, the pain may also occur at rest or at night. The pain usually occurs near the affected joint; however, in some cases, the pain may be referred to other areas. For example, the pain of OA of the hip may actually be felt in the knee.

Joints affected by OA may also be tender to the touch. The level of pain is typically constant over time. Any sudden increases in the level of pain may indicate recent injury or an underlying condition such as gout.

Stiffness—Morning stiffness is a common symptom of osteoarthritis. This stiffness usually resolves within 30 minutes of rising, but it may recur throughout the day during periods of inactivity. Some people note a change in symptoms related to the weather.

Swelling (effusion)—Osteoarthritis may cause a type of joint swelling called an effusion, which results from the accumulation of excess fluid in the joint.

Crackling or grating sensation (crepitus)—Movement of a joint affected by osteoarthritis may cause a crackling or grating sensation called crepitus. This sensation likely occurs because of roughening of the normally smooth surfaces inside the joint.

Bony outgrowths (osteophytes)—Osteoarthritis often causes outgrowths of bone called osteophytes or bone spurs. These bony protuberances can be felt under the skin near joints, and typically enlarge over time.

Symptoms in specific joints—Osteoarthritis does not affect all joints equally. The condition most commonly affects the fingers, knees, hips, and spine; it rarely affects the elbow, wrist, and ankle. Furthermore, it often affects joints on one side of the body differently than the other side.

The diagnosis of osteoarthritis is based on a consideration of several factors, including the characteristic symptoms of osteoarthritis and the results of laboratory tests and x-rays. Formal criteria that are often used to diagnose osteoarthritis in specific joints include the following tests.

Osteoarthritis of the knee—The criteria for OA of the knee include the presence of knee pain plus at least three of the following characteristics:

Age greater than 50 years

Morning stiffness lasting less than 30 minutes

Crackling or grating sensation (crepitus)

Bony tenderness of the knee

Bony enlargement of the knee

No detectable warmth of the joint to the touch

Laboratory tests and x-rays are often used in addition to these criteria.

Osteoarthritis of the hand—The criteria for OA of the hand include the presence of hand pain plus at least three of the following characteristics:

Bony enlargement of at least two or more of 10 selected joints

Bony enlargements of two or more distal interphalangeal (DIP) joints

Fewer than three swollen metacarpophalangeal (MCP) joints

Deformity of at least one of the ten selected joints

Osteoarthritis of the hand can often be diagnosed on the basis of these criteria alone, and laboratory tests and x-rays may be unnecessary.

Osteoarthritis of the hip—The diagnosis of OA of the hip relies on the results of laboratory tests and x-rays. The criteria include the presence of hip pain plus at least two of the following characteristics:

A normal erythrocyte sedimentation rate (ESR)

The presence of bony outgrowths (osteophytes) on x-rays

The presence of joint space narrowing on x-rays, indicating a loss of cartilage

Laboratory tests—Laboratory tests may be recommended to help diagnose osteoarthritis by ruling out conditions with similar symptoms.

X-rays—X-rays are often helpful for tracking the status of osteoarthritis over time, but x-rays may appear normal during the early stages.

Thus, in one aspect, donors are selected that have a confirmed diagnosis of osteoarthritis in a least one joint. In certain embodiments, donors are selected based the diagnosis of osteoarthritis of the hip. In another aspect donors are selected based on the diagnosis of osteoarthritis of the knee. In another aspect donors are selected based on the diagnosis of osteoarthritis of the hand. In another aspect donors are selected based on the diagnosis of generalized osteoarthritis. In another aspect donors are selected based on the diagnosis of osteoarthritis of more than one joint.

In one embodiment of osteoarthritis donor selection, donor is selected to be less than 45 years old. In another aspect the donor is selected to be less than 55 years old. In another aspect the donor is selected to be less than 65 years old. In one embodiment the donor is selected to be older than 45 years old.

In another aspect of the claimed methods, the antibodies of the Disclosure are obtained from human B cells derived from pre-selected donors who have been diagnosed with one or more symptoms of multiple sclerosis (MS). In one aspect, the donor is in stable remission, meaning that one or more of the patient's symptoms of MS have progressively decreased, or have remained substantially unchanged for at least about three months, about six months, or about twelve months or longer.

Symptoms of MS include neuronal demyelination, characterized by alternating relapsing/remitting phases, which correspond to episodes of neurologic dysfunction lasting several weeks followed by substantial or complete recovery. Periods of remission grow shorter over time. The basic hallmark of MS is the demyelinated plaque with reactive glial scar formation, seen in the white matter tracts of the brain and spinal cord. Demyelination is linked to functional reduction or blockage in neural impulse conduction. Axonal transection and death is also observed in MS patients. Pathological studies show the majority of involvement limited to the optic nerves, periventricular white matter, brain stem and spinal cord. The effects of these CNS deficiencies include the acute symptoms of diplopia, numbness and unsteady gait, as well as chronic symptoms such as spastic paraparesis and incontinence.

A typical presentation of MS involves an initial course, running for several years to more than a decade, manifest by episodes of relapse followed by remission. Relapses often follow an episode of a viral infection of the upper respiratory system or gastrointestinal tract. In about one third of MS patients, this disease evolves into a progressive course termed “secondary progressive MS.” In a minority of patients, progressive neurologic deterioration without remission occurs from the onset of disease, and this is called “primary progressive MS.” The pathophysiologic and genetic causes underlying primary versus secondary progressive MS remain unclear.

In another aspect of the claimed methods, the antibodies of the Disclosure are obtained from human B cells derived from pre-selected donors who have been diagnosed with one or more symptoms of rheumatoid arthritis (RA). In one aspect, the donor is in stable remission, meaning that one or more of the patient's symptoms of RA have progressively decreased, or have remained substantially unchanged for at least about three months, about six months, or about twelve months or longer.

Symptoms of RA include the presence of swollen and tender joints with significant pain both systemically and localized in the affected joints. Among the characteristic hallmarks of the disease is the presence of cartilage destruction, bone erosions, periarticular osteoporosis and generalized bone loss resulting in increased prevalence of osteoporotic fractures. Some of the disease mechanisms responsible for focal bone loss may be similar to processes of generalized osteoporosis and associated with osteoclast activation. Generalized bone loss in patients with RA will occur as a result of the systemic and local activation of the immune-system and the presence of proinflammatory cytokines with an ability to stimulate resorption of bone. In turn these mediators accelerate bone turnover and systemic bone loss. In addition, bone loss also takes place focally as a consequence of the arthritic disease process. Another significant contributing factor to systemic bone loss in RA is the common therapeutic use of glucocorticoids, which in addition to their well-documented anti-inflammatory effect also have a stimulating effect on bone resorption.

Donor's sera can be used for an initial determination of their seropositivity to full length osteopontin, since the specificity and long-term maintenance of the adaptive immune responses (even years after the last exposure to this antigen) allows a qualitative determination that is sufficient for the initial selection of suitable donors. Additionally peripheral blood is usually easier to obtain, and can be easily, stored, and monitored for the serological response against osteopontin. Initial screening can be completed using established screening methodology such ELISA which are simple to set up and readily amenable to high throughput analysis. Additionally, antibody secreting cells can be obtained from other sources, including, for example spleen, lymph nodes, bone marrow, or lymphocytes from the osteoarthritic joints of donors. These cells can then be isolated and immortalized as described below.

In the clinical context, the choice of the tissue or the organ from which the antibody secreting cells are isolated can be dictated by the availability of the cells in order to obtain sufficient cells for performing the whole process. Given that cells may be obtained from human clinical samples in relatively small quantities and/or prepared in locations different from where the immortalization methods may be performed, the cells can be conveniently obtained from frozen samples and/or from samples obtained from a number of individuals that have been pooled to provide enough starting material. Additionally, samples may be collected from the sample donors over a duration of several months in order to identify differences in the pattern of antibodies produced during periods of remission compared to periods of flare-ups.

### Isolation of Antibody Secreting Cells

Peripheral blood mononuclear cells (PBMCs) can be isolated from blood or lymphatic tissues using standard separation techniques such as gradient centrifugation. For example, starting from 5-50 ml of peripheral blood, approximately 10-100 million of PBMCs can be purified, a number of cells that should allow obtaining a sufficiently large population of antibody-secreting cells to be screened after being immortalized using the methods of the Disclosure.

After the isolation of PBMCs from the biological samples, a specific selection of antibody-secreting cells can be performed based on the expression of cell surface markers, or if appropriate, on the expression of other proteins, as well as the proliferation activity, or the metabolic and/or morphological status of the cells.

In particular, various approaches for the purification of antibody-secreting cells from human samples are established in the literature and typically make use of different means and conditions for positive or negative selection. These approaches enable the efficient selection of B cells, and more specifically memory B cells, from other cells. Specific protocols can be found in the literature (see for example, Callard R and Kotowicz K “Human B-cell responses to cytokines” in Cytokine Cell Biology: A practical Approach. Balkwill F. (ed.) Oxford University Press, 2000, pg. 17-31).

The cell selection is typically performed using labeled antibodies that bind specifically to one or more B cell specific cell surface proteins. Once bound to the B cell, the labeled cells can be either coupled to solid supports (e.g., to paramagnetic microbeads or plastic plates) and separated by affinity separation, or if the antibody is labeled with a fluorohore, the cells can be separated using commercially available fluorescence-activated cell sorters (FACS). Magnetic based separations for example can be performed using commercially available magnetic separation systems (i.e., MACS® Cell separation Technology, Milteny Biotech) which enable the automated, reproducible cell incubation and washing of the cells. These systems are well validated, and are capable of separating up to 10¹¹ cells in less than 30 minutes and can isolate extremely rare clones that retain viability and can be subsequently grown and manipulated.

Useful B cell markers that may be used for the selection of B cells include for example CD19, CD27, and/or CD22. Additional selections may also be completed to separate B cells expressing (or not expressing) specific antibody isotypes, such as IgM or IgE prior immortalization.

CD19 encodes a cell surface molecule which assembles with the antigen receptor of B lymphocytes in order to decrease the threshold for antigen receptor-dependent stimulation. CD19 is expressed on follicular dendritic cells and B cells and is present on B cells from earliest recognizable B-lineage cells during development to B-cell blasts but is lost on maturation to plasma cells.

CD22 which is a B-cell restricted transmembrane protein that controls signal transduction pathways related to antigen recognition and B cell activation is a preferred molecule for the initial B cell selection. Since the CD22 positive population contains cells that express antibodies having different isotypes and specificities, other cell surface markers can be used for further selecting the cells, either before or after the stimulation phase.

Alternatively, a specific enrichment of antibody-secreting cells can be obtained by applying a CD27-based selection in addition to the CD22-based selection. CD27 is known to be a marker for human B cells that have somatically mutated variable region genes (Borst J et al., 2005 Curr. Opin. Immunol. 17(3):275-81). Additional markers such as CD5, CD24, CD25, CD86, CD38, CD45, CD70, or CD69 can also be used to either deplete or enrich for the desired population of cells. Thus, depending on the donor's history of exposure to the antigen (e.g., viral, bacterial, parasite), the antibody titer, a decision can be taken as to whether to use total, CD22 enriched B cells, or further enriched B cell subpopulations such as CD27 positive B cells.

In one embodiment of any of the claimed methods, nucleic acids encoding all or part of the heavy and light chains of the subject antibodies may be obtained by recombinant DNA amplification from nucleic acids derived from B cells, obtained using any of the B cell isolation methods described above, or known in the art. Direct amplification of DNA or RNA from single cell clones, or cell colonies has the advantage of avoiding the time delay required to immortalize the cells, as described more fully below.

The sequences of the antibodies present within the isolated B cells can be readily determined by isolating nucleic acids encoding these antibodies using recombinant DNA technologies that are known in the literature (Poul M A et al., (1995) Eur J Immunol. 25(7):2005-9; Jovelin F et al., (1995) Biotechniques. 19(3):378-80; Heinrichs A et al., (1995) J Immunol Methods. 178(2):241-51; Essono S et al., (2003) J Immunol Methods. 279(1-2):251-66).

Once the full length antibody sequences have been obtained the resulting sequences can be cloned into suitable expression vectors, and further characterized and screened using any of the methods described herein.

### Methods for Immortalization Memory B Cells

In one aspect, of any of the claimed methods, the B-cells isolated from the patient can be immortalized to facilitate subsequent analysis and screening. In any of methods of the disclosure, antibody secreting cells can be immortalized by transformation with a viral immortalizing agent in the presence of a polyclonal B cell activator. In one preferred aspect, the viral immortalizing agent is the Epstein-Barr virus (EBV), and the polyclonal B cell activator is an immunostimulatory nucleic acid molecule.

Preferred viral immortalizing agents, include lymphotropic viruses grouped in the gamma class of herpesvirus. Members of this virus family infect lymphocytes in a species-specific manner, and are associated with lymphoproliferative disorders and the development of several malignancies (Nicholas J, (2000) Mol Pathol. 53(5):222-37). Particularly preferred for use in the present disclosure as a viral immortalizing agent is the Epstein-Barr Virus (EBV).

EBV (also known as herpesvirus 4), and HHV-8 (human herpesvirus 8, also known as KSHV, Kaposi's Sarcoma associated Herpesvirus) infect and immortalize human lymphocytes. MHV-68 (murine herpesvirus 68), HVS (herpesvirus Samiri), RRV (Rhesus Rhadinovirus), LCV (primate Lymphocryptovirus), EHV-2 (Equine Herpesvirus 2) HVA (Herpesvirus Ateles), and AHV-I (Alcelaphine Herpesvirus 1) are other oncogenic, lymphotropic herpesvirus having some common genetic features conserved amongst them and similar pathogenic effects in different mammalian host cells. These viruses can be used whenever the methods of the Disclosure are applied on antibody-secreting cells obtained from such mammals.

Typically, suitable EBV viral supernatants that can be used in the methods of the Disclosure can be produced using common techniques for infecting human or rodents cell cultures with any of the EBV laboratory, partially deleted, or recombinant EBV strains (as well as mini-EBV and other EBV-based vectors), and separating the infected cells from the EBV-enriched supernatants. See generally: Amoli, et al., (2008), Int J Epidemiol. 37 Suppl 1:i41-5; Oh et al., (2003) Cell Prolif. 36(4):191-7; Biddison. (2001) Curr Protoc Cell Biol.; Chapter 2:Unit 2.4; Tosato & Cohen (2007) Curr Protoc Immunol. Chapter 7:Unit 7.22; Chang et al., (2006) Cell Prolif.; 39(6):457-69; Bass and Darke, (2004); Cell Prolif.; 37(6):443-4; Radons J et al., (2005) J Immunol Methods.; 303 (1-2):135-41 and U.S. Pat. No. 5,798,230).

Additionally immortalization can be accomplished using recombinant DNA constructs that contain specific viral proteins obtained from the virus used to immortalize B cells (Kilger et al., (1998) EMBO J. 17(6):1700-9; Wang L, et al., (2006) Cancer Res. 1; 66(7):3658-66). Similar vectors containing viral genes can be transduced into cells, sometimes making use of retroviral systems or virus-like particles into packaging cell lines which provide all the necessary factors in trans for the formation of such particles, can also be used in the methods of the Disclosure.

EBV-mediated immortalization of B cells requires the expression of the cell surface receptor CD21 which is considered as the main EBV receptor. CD21 is present on most B cell subpopulations and regulates B cell responses by forming a complex with CD19 and the B cell antigen receptor (Fearon D and Carroll M, (2000) Annu. Rev. Immunol.; 18:393-422). However, CD21 is lost from the cell surface following extensive activation of cells, and as they transform in to plasma cells. Thus, the ability to transform cells with EBV may be aided by the addition of B cell stimulating agents, but the conditions must ensure that CD21 is maintained on the cell surface, allowing EBV immortalization at high efficiency.

Thus human B cells can be efficiently immortalized using EBV supernatants if first selected for CD22 expression, then stimulated for an appropriate time (from about 2 days to about 4 days) and with an appropriate combination of stimulating agents (a polyclonal activator, such as an immunostimulatory nucleic acid molecule and irradiated allogenic PBMCs), and finally selected on the basis of a preferred isotype (IgG positive or enriched; IgM negative or depleted).

Typically the immortalization phase can last between 1 and several hours, up to 2-4 days, and, in the case of EBV at least, 4 hours can be sufficient to establish polyclonal populations of lymphoblasts (large viable cells, as measured by microscopy and or FACS; that provide immortalized antibody-secreting cells. The amount of EBV supernatant to be added to the cell culture can be that commonly indicated in the literature (10%, 20%, 30%, or more), though typically conditions in which the amount of EBV supernatant is relatively high (50% V/V) but the exposure is relatively short (from about 4 to about 24 hours) are preferred.

Important aspects of this method include the careful control of culture conditions and the use of a cell feeder layer, which is required for culturing the antibody-secreting cells during and following the immortalization phase, when cells are cultured at low density. The feeder layer can be constituted by irradiated non-/allogeneic peripheral blood cell preparations, lymphoblastoid or fibroblast cell lines, cord blood lymphocytes, or different types of embryonic cells. An example of a cell line having such properties is EL4-B5, which is a mutant EL4 thymoma cell line that efficiently supports the growth and the proliferation of B cells (Ifversen P et al., (1993); Hum Antibodies Hybridomas. 4(3):115-23).

Additionally immortalization requires the addition of specific B cell growth promoting agents—Polyclonal activators in the cell culture medium, as more fully described below.

### Polyclonal Activators

Toll Like Receptors (TLBs) are pattern recognition receptors of the innate immune system expressed on a variety of cells including dendritic cells and B cells (Medzhitov & Janeway. (2000) Trends Microbiol.; 8(10):452-6; Medzhitov & Janeway. (2002) Annu. Rev. Immunol. 20:197-216).

TLR agonists include microbial products and synthetic compounds. Preferred polyclonal activators are; agonists of the Toll Like Receptors which are expressed on memory B cells, such as TLR-7, TLR-9 and TLR-10 (Bernasconi et al. (2003) Blood. 1; 101(11):4500-4). Such molecules may be of microbial or cellular origin or synthetic.

Unmethylated DNA oligonucleotides (immunostimulatory nucleic acid molecules) are TLR-9 agonists. They stimulate dendrite cell maturation and activate B cell proliferation and differentiation polyclonally, i.e., irrespective or the antibody specificity (Krieg et al. (1995), J Clin Immunol.; 15(6):284-92). The biological effect of an immunostimulatory nucleic acid molecule is dependent on specific sequences and chemical modifications (Krieg (2002) Trends Immunol. 23(2):64-5). Immunostimulatory nucleic acid molecules can be used as polyclonal activators, and examples of suitable activators are immunostimulatory nucleic acid molecules such as CpG 2006 (5′-TCGTCGTTTTGTCGTTTTGTCGTT-3′; Hartmann et al. (2000) J Immunol. 15; 164(2):944-53) and other oligonucleotide sequences that trigger TLR-9.

4-Imidazoquinoline compounds, such as R-848 (resiquinol), trigger TLR-7 and TLR-8 and stimulate dendritic cell maturation (Hemmi et al. (2002) Nat Immunol. 3(2):196-200). Such compounds can be used as polyclonal activators with the disclosure e. g. R-848 (and its analogs) and other synthetic compounds that trigger TLR-7 and TLR-8, including but not limited to: imiqulinol, loxoribine, and guanosine analogs (e.g 7-thia-8-oxoguanosine and 7-deazaguanosine).

Other polyclonal activators include other agonists of TLRs, including monoclonal antibodies specific for TLRs, and to other pattern recognition receptors (PRRs) that are expressed on memory B cells. Additional polyclonal activators include 13D40L, BAFF (B-cell activating factor, (Schneider et al. (1999), J Exp Med. 189(11):1747-56) also known as tumor necrosis factor superfamily member 13B, BLyS, or THANK7, antibodies specific for CD40 and other molecules expressed by dendritic cells and activated T cells. In these cases the cells themselves may be used as polyclonal activators.

Polyclonal activators may also include PAMPs pathogen-associated molecular patterns), such as lipopolysaccharide (LPS), peptidoglycans, flagellins, zymosans and other cell wall components found in pathogens. Other available polyclonal activators include loxoribine, heat-killed Acholeplasma ladilawii, heat-killed Listeria monocytogenes, lipoteichoic acids, tripalmitoylated lipopeptides (e.g Pam3CSK4), single-stranded RNA (Diebold et al. (2004) Science. 303 (5663):1529-31), double-stranded RNA, poly(T:5), bacterial DNAs, etc. A detailed list of TLR agonists can be ford in Takeda et al. (2004) Mol Immunol. 40(12):861-8). Some activators are not preferred for use with human B cells e.g., LPS.

In a particularly preferred aspect, CpG 2006 is used as the polyclonal activator. Commercial suppliers of suitable polyclonal activators include Invitrogen (USA) & Microsynth, CH).

It is preferable to proceed with the screening assays as soon as possible after seeding the pools of cells, and without the need to eliminate B cell growth promoting agents (or any other element present in cell culture supernatant) by establishing appropriate conditions that do not elicit problems in the screening assays, for example by washing the cells, or by changing the cell culture medium The antibody-producing cells are isolated, stimulated, and immortalized according to the methods of the Disclosure, and then can be kept in bulk cultures for a variable number of days (e.g., from 1 up to 10 days, or for longer periods of time such 2-4 weeks) before being subdivided into several pools, each representing a population of cells, that are cultured separately (e.g., in 6-, 12-, 24-, 32-, or 96-well plates).

The population of immortalized B cells may then be cloned and screened as described below to isolate clones of antibody-secreting cells, with the desired characteristics.

### Antibody Screening

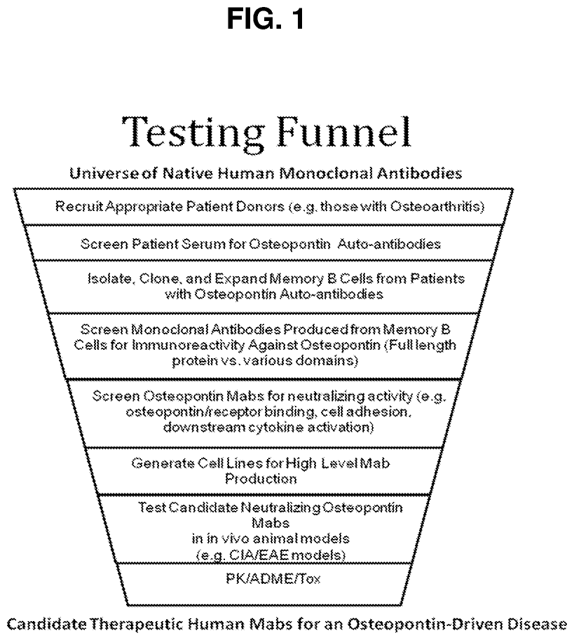

For any of the claimed methods, the antibody screening step may be carried out by ELISA, by staining of tissues or cells (including transfected cells), antigen microarrays, mass spec analysis, specific neutralization assays or one of a number of other methods known in the art for identifying desired antigen specificity. As outlined in FIG. 1, suitable antibodies may be obtained by a series of sequential selectivity filters to narrow down the number of candidate antibodies to a limited set of antibodies that all exhibit the required attributes. The assay may select on the basis of simple antigen recognition, or may select on the additional basis of a desired function e.g., to select neutralizing antibodies rather than just antigen-binding antibodies, to select antibodies that can change characteristics of targeted cells, such as their signaling cascades, their shapes their grown rate, their capability of influencing other cells, their response to the influence by other cells or by other reagents or by a change in conditions, their differentiation status, etc.

Antibody specificity can be further characterized by determining the ability of the antibodies to inhibit the binding of osteopontin to CD44, and integrins αᵥβ₃, αᵥβ₁, αᵥβ₅, α₉β₁ and α₄β₁. In this case, either purified receptor extracellular domains or whole cells expressing these receptors could be coated onto microtiter wells.

In another aspect antibody specificity can be further characterized by determining the ability of the antibodies to selectively bind to different splice variants and proteolytic forms of osteopontin. In one aspect, of any of the claimed methods, antibodies may be identified that selectively bind to OPN-a, OPN-b, or OPN-c, respectively. In another aspect of any of the claimed methods, antibodies may be identified that selectively bind to any of a number of different combinations of OPN-a, OPN-b, or OPN-c. In another aspect of any of the claimed methods, antibodies may be identified that selectively bind to osteopontin-L. In another aspect of any of the claimed methods, antibodies may be identified that selectively bind to osteopontin-R. In another aspect of any of the claimed methods, antibodies may be identified that selectively bind to osteopontin-166. In one aspect, of these methods, selectively means that the antibodies bind to one form of osteopontin with at least about a 2 fold higher affinity than the binding of that antibody to another form of osteopontin. Relative affinities may be determined by methods known in the art including cross competition assays, Biacore© or Kinexa analysis.

In another aspect of any of these methods, the antibodies are screened for selective binding to osteopontin splice variants OPN-a, OPN-b, or OPN-c, or any combination of these splice variants. In another aspect of any of these methods, the antibodies are screened for binding to osteopontin-R. In another aspect of any of these methods, the antibodies are screened for binding to osteopontin-L. In another aspect of any of these methods, the antibodies are screened for binding to osteopontin-166.

In another aspect of any of these methods, the antibodies are screened for binding to the CD44 binding site of osteopontin. In another aspect of any of these methods, the antibodies are screened for binding to the αᵥβ₃ binding site of osteopontin. In another aspect of any of these methods, the antibodies are screened for binding to the αᵥβ₁ binding site of osteopontin. In another aspect of any of these methods, the antibodies are screened for binding to the αᵥβ₅ binding site of osteopontin. In another aspect of any of these methods, the antibodies are screened for binding to the α₅β₁ binding site of osteopontin. In another aspect of any of these methods, the antibodies are screened for binding to both the αᵥβ₃ binding site of osteopontin and the CD44 binding site osteopontin.

Osteopontin antibody binding can then be further assessed in the presence or absence of media conditioned by the memory B cells, from normal donors, or patients in remission or with active disease.

The ability of the antibodies to block osteopontin mediated cell adhesions could be assessed by coating osteopontin onto microtiter wells and assessing cell attachment (e.g., counting # of adhered cells) in the presence or absence of media conditioned by the memory B cells, or by increasing amounts of the purified antibodies.

The ability of the antibodies to block downstream signaling elicited by osteopontin stimulation of cells could be assessed by coating osteopontin onto a microtiter well and measuring the secretion of various cytokines including MCP1, MIP from the test cells in the presence or absence of media conditioned by the memory B cells, or by increasing amounts of the purified antibodies.

### Cloning of Antigen-Binding Domains

The cloning step for separating individual clones from the mixture of positive cells may be carried out using limiting dilution, micromanipulation, single cell deposition by cell sorting or another method known in the art. Preferably the cloning is carried out using limiting dilution.

The methods of the disclosure produce immortalized B cells that secreted antibodies having desired antigen specificity. The disclosure thus provides an immortalized B cell clone obtainable or obtained by the methods of the disclosure. These B cells can be used in various ways and as a source of monoclonal antibodies, as a source of nucleic acid (DNA or mRNA) encoding a monocional antibody of interest, for delivery to patients for cellular therapy, for research, etc.

The protein sequence of the antibodies secreted by the selected clonal cell cultures can be easily determined by isolating nucleic acids encoding these antibodies using recombinant DNA technologies that are known in the literature (Poul M A et al., (1995) Eur J Immunol. 25(7):2005-9; Jovelin F et al., (1995) Biotechniques. 19(3):378-80; Heinrichs A et al., (1995) J Immunol Methods. 178(2):241-51; Essono S et al., (2003) J Immunol Methods. 279(1-2):251-66).

These technologies can also be used for further structural and functional characterization and optimization of therapeutic antibodies (Kim S J et al., (2005) Mol Cells. 20(1):17-29. Aires da Silva F, et al., (2008) BioDrugs. 22(5):301-14), or for generating vectors allowing the stable in vivo delivery of monoclonal antibodies.

Briefly, mRNA can be prepared from the cell culture and retrotranscribed into a cDNA library, which can be used as a template for a Polymerase Chain Reaction (PCR) including degenerate primers for specifically amplifying full heavy and light chains or only portions of these chains (such as the variable regions). In the case where only the variable regions (responsible of antigen-binding) are isolated, these sequences can be cloned in a vector allowing the fusion of this sequence to constant (Fc) regions of the desired isotype (for example, human IgG). The PCR-amplified DNA fragments can be directly sequenced or cloned, using adaptors or restriction sites, into vectors for sequencing the coding sequence that can be adapted and recloned in other vectors for expressing antibodies as recombinant proteins

The mRNA of the polyclonal or oligoclonal populations of cells can also be used for constructing cDNA libraries specific for antibody-secreting cells of specific isotypes that can be made available, for example, as phage display libraries, bacterial libraries, yeast libraries, or any other format of biological library that can be used for replicating and maintaining DNA, in particular DNA encoding proteins. For instance, a library of recombinant antibody sequences can be generated using the mRNA extracted from one or more oligoclonal populations of cells, used for producing antibodies in bacterial or eukaryotic host cells, and then for screening such antibodies at the scope of identifying one or more antibodies that have a desired antigen specificity and/or biological activity.

Once cloned and characterized, the antibodies can be expressed as recombinant proteins in prokaryotic organisms (e.g., E. coli; Sorensen H and Mortensen K, (2005) Microb Cell Fact. 4(1):1; Venturi et al., 2002 J Mol Biol.; 315(1):1-8), plants (Ma J K et al., 2005 Vaccine. 23(15):1814-8), or eukaryotic cells, in particular human, rodent, or other eukaryotic cell lines (e.g., CHO, COS, HEK293) that allow a high level of expression as transient or stable transformed cells. This would be required in particular when the characterization of the antibodies has to be performed using more sophisticated assays, including in vivo assays. The host cells can be further selected on the basis of the level of recombinant expression of the cloned monoclonal antibody.

At this scope, the cloned antibody sequences can be modified using PCR or other recombinant DNA technologies at the DNA level only (e.g., eliminating or adding restriction sites, optimizing the codon usage, adapting transcription and/or translation regulatory sequences) or at both the DNA and protein level (e.g., adding other protein sequences or modifying internal amino acids). Moreover, fragments (Fv, Fab, F(ab)′ or F(ab)″) or fusion proteins based on these antibodies can be produced using recombinant DNA technologies.

For example, recombinant antibodies can also be modified at the level of structure and/or activity by choosing a specific Fc region to be fused to the variable regions (Furebring C et al., 2002), by adding stabilizing peptide sequences, (WO 01/49713), by generating recombinant single chain antibody fragments (Gilliland L K et al., 1996), or by adding radiochemicals or polymers to chemically modified residues (Chapman A et al., 1999).

Different vector systems have been used for generating stable pools of transfected cell lines (Aldrich T L et al., (2003) Biotechnol Prog. 19(5):1433-8; Bianchi A and McGrew J T, (2003) Biotechnol Bioeng. 84(4):439-44). High level, optimized, stable expression of recombinant antibodies has been achieved (Schlatter S et al., (2005) Biotechnol Prog. 21(1):122-33; Dinnis D and James D, (2005) Biotechnol Bioeng. 91(2):180-9; Kretzmer G, (2002) Microbiol Biotechnol. 59(2-3):135-42), thanks to the optimization of cell culture conditions (Grunberg J et al., (2003); Biotechniques. 34(5):968-72; Yoon S K et al., (2004) Biotechnol Prog. 20(6):1683-8) and by selecting or engineering clones with higher levels of antibody production (Bohm E et al., (2004) Biotechnol Bioeng. 88(6):699-706; Borth N, (2002) Biotechnol Bioeng. 80(1):93-9).

The purification of non-/recombinant antibodies from cell cultures can be performed using the technologies described in the literature (Horenstein A L et al., (2003) J Immunol Methods. 275(1-2):99-112). However, clinical development and use should be based on the characterization of the antibody pharmacokinetics and pharmacodynamics (Lobo E et al., (2004) J Pharm Sci. 93(11):2645-68) and compliancy to international requirements for the production and quality control of murine, human and engineered monoclonal antibodies for therapeutic and in vivo diagnostic use in humans (EUDRA document 3AB4a).

### Screening of Animal Models of Osteopontin Related Diseases.

In another aspect of the disclosure, the disclosure includes a method for selecting a non human auto-antibody to osteopontin from a non human subject. In one aspect, the subject has an osteopontin related disease, or the corresponding animal model for that disease, and/or is a transgenic organism with an osteopontin knockout (Rittling & Denhart, Exp. Nephrol. 7(2) 103-13 (1999). In one aspect, the subject is a mouse, rat, dog, rabbit chicken, guinea pig or primate.

Representative animal models of OA include for example chemically-induced (intra-articular) models such as iodoacetate mediated OA in chicken, papain mediated OA in guinea pig & mouse, chymopapain mediated OA in dog, collagenase mediated OA in mouse and TGF-ß mediated OA in mouse. Physically induced animal models of OA include anterior cruciate ligament transection in dogs and rabbit, meniscectomy in rabbit & guinea pig, immobilization in rabbit, dog and patellar contusion in rabbit. Spontaneously occurring animal models of OA include Age/obesity mediated OA in Hartley guinea pig, transgenic mice with type IX or II collagen mutations and in bred dogs with hip dysplasia.

Many animal models for human MS have been described in mice, rats, rabbits, guinea pigs, marmosets, and rhesus monkeys. (Friese et al., Brain 129 1940-1952 (2006)) Representative animal models for MS include experimental allergic (or autoimmune) encephalomyelitis (EAE), Semliki Forest virus (SFV) mediated disease, mouse hepatitis virus (MHV) mediated disease, and Theiler's murine encephalomyelitis virus (TMEV) mediated disease.

One of skill in the art will recognize that different forms of EAE can be produced by immunization with different antigens, such as myelin basic protein, proteolipid protein, or myelin oligodendrocyte glycoprotein (MOG), and that by using different animal genetic backgrounds it is possible to mimic either an acute MS-like illness or to produce spontaneous relapsing-remitting forms of EAE that mimic remission in human MS. (Nelson et al., Int. MS J. 11 95-99 (2004)).

Animal models of rheumatoid arthritis include rat adjuvant arthritis, rat type II collagen arthritis, mouse type II collagen arthritis, streptococcal cell wall induced or mycobacterium induced reactive arthritis in Lewis rats, and antigen induced arthritis (AIA) (Vierboom et al., Arthritis Res & Therapy 7 145-154 (2005)). As with other animal models of antigen induced disease, the type of disease is heavily influenced by the genetic background of the animals used. Additionally a number of mouse strains and transgenic organisms spontaneously develop RA-like lesions, as a result of genetic manipulations, including MRL/Ipr mice, HLA-B27 transgenic rats, B2 microglobulin deficient mice, and mice transgenic for the VB6T cell receptor (TCR) crossed with NOD mice (Bendele J. Musculoskel Neur. Interact 1(4) 377-385).

Such animal models may be used in any of the claimed methods to identify an antibody to osteopontin capable of selectively blocking the pro-inflammatory functions of osteopontin within disease tissues.

In one embodiment of the claimed methods, the antibodies of the disclosure are obtained from non-human B-cells derived from animal subjects that have an animal model disease of OA, RA or MS. In one aspect, animals are selected that are in remission, or who display reduced or less severe symptoms compared to the normal disease severity. Such antibodies may be screened and cloned using any of the previously disclosed methods for antibody screening and cloning as provided herein.

In one aspect, such a non human antibody will be humanized to create a therapeutic antibody suitable for use in humans.

“Humanized” forms of non-human (e.g., murine) antibodies generally refers to chimeric antibodies which contain minimal sequence derived from non-human immunoglobulin. For the most part, humanized antibodies comprise a human recipient antibody in which hypervariable region residues of the recipient are replaced by hypervariable region residues from a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues which are not found in the recipient antibody or in the donor antibody. These modifications are made to further refine antibody performance. In general, the humanized antibody will comprise substantially all of at least one, and typically two, variable domains, in which all or substantially all of the hypervariable regions correspond to those of a non-human immunoglobulin and all, or substantially all, of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522 525 (1986); Reichmann et al., Nature 332:323 329 (1988); and Presta, Curr. Op. Struct. Biol. 2:593 596 (1992).

### Methods of Use

### Therapeutic Uses

For therapeutic applications, the antibodies, identified by the methods of the present disclosure can be administered to a mammal, preferably a human, in a pharmaceutically acceptable dosage form such as those discussed herein, including those that can be administered to a human intravenously as a bolus or by continuous infusion over a period of time, by intramuscular, intraperitoneal, intra-cerebrospinal, subcutaneous, intra-articular, intrasynovial, intrathecal, oral, topical, or inhalation routes. Antibodies identified by the methods of the present disclosure also can be suitably administered by intra tumoral, peritumoral, intralesional, or perilesional routes, to exert local as well as systemic therapeutic effects. The intraperitoneal route is expected to be particularly useful, for example, in the treatment of ovarian tumors.

For the prevention or treatment of disease, the appropriate dosage of a therapeutic protein will depend on the type of disease to be treated, as defined above, the severity and course of the disease, whether the protein is administered for preventive or therapeutic purposes, previous therapy, the patient's clinical history and response to the protein, and the discretion of the attending physician. The antibody is suitably administered to the patient at one time or over a series of treatments.

In one aspect, the present disclosure includes an anti-osteopontin antibody or an antibody fragment derived therefrom, wherein said antibody can bind to a function directing region or conformer of osteopontin. In one aspect, the anti-osteopontin antibody selectively inhibits the ability of osteopontin to promote disease progression, while preserving the ability of osteopontin to fulfill its housekeeping functions.

In one aspect, the present disclosure also includes an anti-osteopontin antibody or an antibody fragment derived therefrom, wherein said antibody can inhibit the binding between an integrin recognizing the site of amino acid sequence motif RGD and osteopontin.

In one aspect, the present disclosure also includes an anti-osteopontin antibody or an antibody fragment derived therefrom, wherein said antibody can inhibit the binding between an integrin recognizing the site of amino acid sequence motif SVVYGLR and osteopontin.

In one aspect, the present disclosure also includes an anti-osteopontin antibody or an antibody fragment derived therefrom, wherein said antibody can inhibit the binding between an integrin recognizing the site of amino acid sequence motif ELVTDFTDLPAT and osteopontin.

In one aspect, the present disclosure also includes an anti-osteopontin antibody or an antibody fragment derived therefrom, wherein said antibody can inhibit the binding between αᵥβ₃ integrin and osteopontin.

In one aspect, the present disclosure also includes an anti-osteopontin antibody or an antibody fragment derived therefrom, wherein said antibody can inhibit the binding between αᵥβ₁ integrin and osteopontin.

In one aspect, the present disclosure also includes an anti-osteopontin antibody or an antibody fragment derived therefrom, wherein said antibody can inhibit the binding between αᵥβ₅, integrin and osteopontin.

In one aspect, the present disclosure also includes an anti-osteopontin antibody or an antibody fragment derived therefrom, wherein said antibody can inhibit the binding between α₉β₁, integrin and osteopontin.

In one aspect, the present disclosure also includes an anti-osteopontin antibody or an antibody fragment derived therefrom, wherein said antibody can inhibit the binding between α₄β₁, integrin and osteopontin.

In one aspect, the present disclosure also includes an anti-osteopontin antibody or an antibody fragment derived therefrom, wherein said antibody can inhibit the binding between CD44 and osteopontin.

In one aspect, the present disclosure also includes a therapeutic agent for an osteopontin related disease, wherein said therapeutic agent comprising an antibody, or an antibody fragment derived therefrom made according to any of the methods disclosed herein or an antibody fragment derived therefrom as effective ingredients.

In one aspect, the present disclosure also includes a therapeutic agent for rheumatism, wherein said therapeutic agent comprising an antibody, or an antibody fragment derived therefrom made according to any of the methods disclosed herein. or an antibody fragment derived therefrom as effective ingredients.

In one aspect, the present disclosure also includes a therapeutic agent for rheumatoid arthritis, wherein said therapeutic agent comprising an antibody, or an antibody fragment derived therefrom made according to any of the methods disclosed herein. or an antibody fragment derived therefrom as effective ingredients.

In one aspect, the present disclosure also includes a therapeutic agent for osteoarthritis, wherein said therapeutic agent comprising an antibody, or an antibody fragment derived therefrom made according to any of the methods disclosed herein. or an antibody fragment derived therefrom as effective ingredients.

In one aspect, the present disclosure also includes a therapeutic agent for multiple sclerosis, wherein said therapeutic agent comprising an antibody, or an antibody fragment derived therefrom made according to any of the methods disclosed herein. or an antibody fragment derived therefrom as effective ingredients.

In another aspect, the disclosure includes a method for therapeutically treating an osteopontin related disease, characterized in administering an antibody, or an antibody fragment derived therefrom, made according to any of the methods disclosed herein from a patient with osteoarthritis.

In one embodiment of this method the disclosure includes a method for therapeutically treating rheumatoid arthritis, characterized in administering an antibody, or an antibody fragment derived therefrom, made according to any of the methods disclosed herein from a patient with osteoarthritis.

In one embodiment of this method the disclosure includes a method for therapeutically treating osteoarthritis, characterized in administering an antibody, or an antibody fragment derived therefrom, made according to any of the methods disclosed herein from a patient with osteoarthritis.

In one embodiment of this method the disclosure includes a method for therapeutically treating multiple sclerosis, characterized in administering an antibody, or an antibody fragment derived therefrom, made according to any of the methods disclosed herein from a patient with osteoarthritis.

In other aspects, this disclosure includes the use of an antibody, or an antibody fragment derived therefrom, made according to any of the methods disclosed herein for the manufacture of a medicament for treating an osteopontin related disease

Also included in the disclosure is the use of an antibody, or an antibody fragment derived therefrom, made according to any of the methods disclosed herein for the manufacture of a medicament for treating rheumatism.

Also included in the disclosure is the use of an antibody, or an antibody fragment derived therefrom, made according to any of the methods disclosed herein for the manufacture of a medicament for treating rheumatoid arthritis.

Also included in the disclosure is the use of an antibody, or an antibody fragment derived therefrom, made according to any of the methods disclosed herein for the manufacture of a medicament for treating osteoarthritis.

Also included in the disclosure is the use of an antibody, or an antibody fragment derived therefrom, made according to any of the methods disclosed herein for the manufacture of a medicament for treating multiple sclerosis.

Also included in the disclosure are specific methods of treating osteopontin related diseases of the nervous system. Specifically, the present disclosure also relates to the use of antibodies to osteopontin, to selectively block osteopontin's role in enhancing cell mediated autoimmunity that occurs in a neurologic diseases, including demyelinating diseases of the CNS or PNS, neuropathies and neurodegenerative diseases.

In accordance with the present disclosure, anti-osteopontin antibodies may also be for treatment and/or prevention of neurologic diseases. In one aspect, the antibodies may be used for neuroprotection, nerve myelination and the generation or re-generation of myelin producing cells. The disclosure further provides for the manufacture of a medicament for treatment and/or prevention of a neurologic disease, as well as pharmaceutical compositions comprising anti-osteopontin antibodies.

### Multiple Sclerosis

Multiple Sclerosis (MS) is an inflammatory demyelinating disease of the central nervous system (CNS) that takes a relapsing-remitting or a progressive course. MS is not the only demyelinating disease. Its counterpart in the peripheral nervous system (PNS) is chronic inflammatory demyelinating polyradiculoneuropathy (CIDP). In addition, there are acute, monophasic disorders, such as the inflammatory demyelinating polyradiculoneuropathy termed Guillain-Barré syndrome (GBS) in the PNS, and acute disseminated encephalomyelitis (ADEM) in the CNS. Both MS and GBS are heterogeneous syndromes. In MS different exogenous assaults together with genetic factors can result in a disease course that finally fulfils the diagnostic criteria. In both diseases, axonal damage can add to a primarily demyelinating lesion and cause permanent neurologic deficits.

In accordance with the present disclosure, anti-osteopontin antibodies may also be for treatment and/or prevention of axonal damage.

In accordance with the present disclosure, anti-osteopontin antibodies may also be for treatment and/or prevention of relapse.

Clinical problems observed in MS patients may include disturbances in visual acuity, sometimes culminating in blindness; double vision; motor disturbances affecting walking and use of the hands; uncoordination; bowel and bladder incontinence; spasticity; and sensory disturbances including loss of touch, pain, temperature and proprioception. The pathology of MS lies entirely in the central nervous system and is characterized by a classic picture of inflammation surrounding venules and extending into the myelin sheath. In accordance with the present disclosure, anti-osteopontin antibodies may also be for treatment and/or prevention of neuronal inflammation.

Trauma is an injury or damage of the nerve. It may be spinal cord trauma, which is damage to the spinal cord that affects all nervous function that is controlled at and below the level of the injury, including muscle control and sensation, or brain trauma, such as trauma caused by closed head injury.

In accordance with the present disclosure, anti-osteopontin antibodies may also be for treatment and/or prevention of neuronal trauma associated with injury or damage to nerve tissue.

Cerebral hypoxia is a lack of oxygen specifically to the cerebral hemispheres, and more typically the term is used to refer to a lack of oxygen to the entire brain. Depending on the severity of the hypoxia, symptoms may range from confusion to irreversible brain damage, coma and death.

In accordance with the present disclosure, anti-osteopontin antibodies may also be for treatment and/or prevention of cerebral hypoxia.

Stroke is usually caused by ischemia of the brain. It is also called cerebrovascular disease or accident. It is a group of brain disorders involving loss of brain functions that occur when the blood supply to any part of the brain is interrupted. The brain requires about 20% of the circulation of blood in the body. The primary blood supply to the brain is through 2 arteries in the neck (the carotid arteries), which then branch off within the brain to multiple arteries that each supply a specific area of the brain. Even a brief interruption to the blood flow can cause decreases in brain function (neurologic deficit). The symptoms vary with the area of the brain affected and commonly include such problems as changes in vision, speech changes, decreased movement or sensation in a part of the body, or changes in the level of consciousness. If the blood flow is decreased for longer than a few seconds, brain cells in the area are destroyed (infarcted) causing permanent damage to that area of the brain or even death.

A stroke affects about 4 out of 1,000 people. It is the 3rd leading cause of death in most developed countries, including the U.S. The incidence of stroke rises dramatically with age, with the risk doubling with each decade after age 35. About 5% of people over age 65 have had at least one stroke. The disorder occurs in men more often than women.

As mentioned above, a stroke involves loss of brain functions (neurologic deficits) caused by a loss of blood circulation to areas of the brain. The specific neurologic deficits may vary depending on the location, extent of the damage, and cause of the disorder. A stroke may be caused by reduced blood flow (ischemia) that results in deficient blood supply and death of tissues in that area (infarction). Causes of ischemic strokes are blood clots that form in the brain (thrombus) and blood clots or pieces of atherosclerotic plaque or other material that travel to the brain from another location (emboli). Bleeding (hemorrhage) within the brain may cause symptoms that mimic stroke.

The most common cause of a stroke is stroke secondary to atherosclerosis (cerebral thrombosis). Atherosclerosis (“hardening of the arteries”) is a condition in which fatty deposits occur on the inner lining of the arteries, and atherosclerotic plaque (a mass consisting of fatty deposits and blood platelets) develops. The occlusion of the artery develops slowly. Atherosclerotic plaque does not necessarily cause a stroke. There are many small connections between the various brain arteries. If the blood flow gradually decreases, these small connections will increase in size and “by-pass” the obstructed area (collateral circulation). If there is enough collateral circulation, even a totally blocked artery may not cause neurologic deficits. A second safety mechanism within the brain is that the arteries are large enough that 75% of the blood vessel can be occluded, and there will still be adequate blood flow to that area of the brain.

A thrombotic stroke (stroke caused by thrombosis) is most common in elderly people, and often there is underlying atherosclerotic heart disease or diabetes mellitus. This type of stroke may occur at any time, including at rest. The person may or may not lose consciousness.

Strokes caused by embolism (moving blood clot) are most commonly strokes secondary to a cardiogenic embolism, clots that develop because of heart disorders that then travel to the brain. An embolism may also originate in other areas, especially where there is atherosclerotic plaque. The embolus travels through the bloodstream and becomes stuck in a small artery in the brain. This stroke occurs suddenly with immediate maximum neurologic deficit. It is not associated with activity levels and can occur at any time. Arrhythmias of the heart are commonly seen with this disorder and often are the cause of the embolus. Damage to the brain is often more severe than with a stroke caused by cerebral thrombosis. Consciousness may or may not be lost. The probable outcome is worsened if blood vessels damaged by stroke rupture and bleed (hemorrhagic stroke).

In accordance with the present disclosure, anti-osteopontin antibodies may also be for treatment of stroke, and ischemia of the brain.

Peripheral Neuropathy is a syndrome of sensory loss, muscle weakness and atrophy, decreased deep tendon reflexes, and vasomotor symptoms, alone or in any combination.

The disease may affect a single nerve (mononeuropathy), two or more nerves in separate areas (multiple mononeuropathy), or many nerves simultaneously (polyneuropathy). The axon may be primarily affected (e.g., in diabetes mellitus, Lyme disease, or uremia or with toxic agents) or the myelin sheath or Schwann cell (e.g., in acute or chronic inflammatory polyneuropathy, leukodystrophies, or Guillain-Barré syndrome). Damage to small unmyelinated and myelinated fibers results primarily in loss of temperature and pain sensation; damage to large myelinated fibers results in motor or proprioceptive defects. Some neuropathies (e.g., due to lead toxicity, dapsone use, tick bite, porphyria, or Guillain-Barré syndrome) primarily affect motor fibers; others (e.g., due to dorsal root ganglionitis of cancer, leprosy, AIDS, diabetes mellitus, or chronic pyridoxine intoxication) primarily affect the dorsal root ganglia or sensory fibers, producing sensory symptoms. Occasionally, cranial nerves are also involved (e.g., in Guillain-Barré syndrome, Lyme disease, diabetes mellitus, and diphtheria). Identifying the modalities involved helps determine the cause.

Neurodegenerative diseases comprise, among others, Alzheimer's disease, Parkinson's disease, Huntington's disease and Amyotrophic Lateral Sclerosis (ALS). In accordance with the present disclosure, anti-osteopontin antibodies may also be for treatment and or prevention of Alzheimer's disease, Parkinson's disease, Huntington's disease and Amyotrophic Lateral Sclerosis (ALS).

Alzheimer's disease is a disorder involving deterioration in mental functions resulting from changes in brain tissue. This includes shrinking of brain tissues, not caused by disorders of the blood vessels, primary degenerative dementia and diffuse brain atrophy. Alzheimer's disease is also called senile dementia/Alzheimer's type (SDAT). It is the most common cause of intellectual decline with aging. The incidence is approximately 9 out of 10,000 people. This disorder affects women slightly more often than men and occurs primarily in older individuals.

The cause is unknown. The neurochemical factors which may participate in generation of the disease include lack of the substances used by the nerve cells to transmit nerve impulses (neurotransmitters), including acetylcholine, somatostatin, substance P, and norepinephrine. Environmental factors include exposure to aluminum, manganese, and other substances. The infectious factors include prion (virus-like organisms) infections that affect the brain and spinal cord (central nervous system). In some families (representing 5 to 10% of cases) there is an inherited predisposition to development of the disorder, but this does not follow strict (Mendelian) patterns of inheritance. The diagnosis is usually made by ruling out other causes of dementia.

Researchers have found that in families that have multiple members with Alzheimer's, there is a particular gene variation which is common to all of those with the disease. The gene, which produces a substance called apolipoprotein E4, is not said to cause the disease, its presence simply increases the chances that the disease may eventually occur. There are many people who have the E4 gene and never become afflicted with Alzheimer's.

The onset is characterized by impaired memory, with progressive loss of intellectual function. There may be mood changes, changes in language capability, changes in gait, and other changes as the disorder progresses. There is a decrease in the size (atrophy) of the tissues of the brain, enlargement of the ventricles (the spaces within the brain), and deposits within the tissues of the brain.

Parkinsons's disease is a disorder of the brain characterized by shaking and difficulty with walking, movement, and coordination. The disease is associated with damage to a part of the brain that controls muscle movement. It is also called paralysis agitans or shaking palsy.

The disease affects approximately 2 out of 1,000 people, and most often develops after age 50. It affects both men and women and is one of the most common neurologic disorders of the elderly. The term “parkinsonism” refers to any condition that involves a combination of the types of changes in movement seen in Parkinson's disease, which happens to be the most common condition causing this group of symptoms. Parkinsonism may be caused by other disorders or by external factors (secondary parkinsonism).

Parkinson's disease is caused by progressive deterioration of the nerve cells of the part of the brain that controls muscle movement (the basal ganglia and the extrapyramidal area). Dopamine, which is one of the substances used by cells to transmit impulses (transmitters), is normally produced in this area. Deterioration of this area of the brain reduces the amount of dopamine available to the body. Insufficient dopamine disturbs the balance between dopamine and other transmitters, such as acetylcholine. Without dopamine, the nerve cells cannot properly transmit messages, and this results in the loss of muscle function. The exact reason that the cells of the brain deteriorate is unknown. The disorder may affect one or both sides of the body, with varying degrees of loss of function.

In addition to the loss of muscle control, some people with Parkinson's disease become severely depressed. Although early loss of mental capacities is uncommon, with severe Parkinson's the person may exhibit overall mental deterioration (including dementia, hallucinations, and so on). Dementia can also be a side effect of some of the medications used to treat the disorder.

Huntington's Disease is an inherited, autosomal dominant neurologic disease. It is uncommon, affecting approximately 1 in 10000 individuals. The disease does not usually become clinically apparent until the fifth decade of life, and results in psychiatric disturbance, involuntary movement disorder, and cognitive decline associated with inexorable progression to death, typically 17 years following onset.

The gene responsible for Huntington's disease is called huntingtin. It is located on chromosome 4p, presenting an effective means of preclinical and antenatal diagnosis. The genetic abnormality consists in an excess number of tandemly repeated CAG nucleotide sequences.

The increase in size of the CAG repeat in persons with Huntington's disease shows a highly significant correlation with age of onset of clinical features. This association is particularly striking for persons with juvenile onset Huntington's disease who have very significant expansion, usually beyond 50 repeats. The CAG repeat length in Huntington's disease families does exhibit some instability that is particularly marked when children inherit the huntingtin gene from affected fathers.

In HD, it is not known how this widely expressed gene, results in selective neuronal death. Further, sequence analysis revealed no obvious homology to other known genes and no structural motifs or functional domains were identified which clearly provide insights into its function. In particular, the question of how these widely expressed genes cause selective neuronal death remains unanswered.

Amyptrophic Lateral Sclerosis, ALS, is a disorder causing progressive loss of nervous control of voluntary muscles because of destruction of nerve cells in the brain and spinal cord. Amyotrophic Lateral Sclerosis, also called Lou Gehrig's disease, is a disorder involving loss of the use and control of muscles. The nerves controlling these muscles shrink and disappear, which results in loss of muscle tissue due to the lack of nervous stimulation. Muscle strength and coordination decreases, beginning with the voluntary muscles (those under conscious control, such as the muscles of the arms and legs). The extent of loss of muscle control continues to progress, and more and more muscle groups become involved. There may be a loss of nervous stimulation to semi-voluntary muscles, such as the muscles that control breathing and swallowing. There is no effect on ability to think or reason. The cause is unknown.

ALS affects approximately 1 out of 100,000 people. It appears in some cases to run in families. The disorder affects men more often than women. Symptoms usually do not develop until adulthood, often not until after age 50.

Ectopic calcification is the inappropriate biomineralization of soft tissues, characterized by the deposition of calcium salts in tissues other than teeth or bone. Ectopic calcification, also termed dystrophic calcification, is characteristic of a number of clinically important diseases such as, for example, atherosclerosis, kidney and renal calculus, arthritis, and the calcification of implanted biomaterial such as prosthetic heart valves (see e.g., U.S. Pat. No. 6,878,168), vascular grafts, LVAD (left ventricular assist devices), contact lenses and a total artificial heart. Ectopic calcifications are typically composed of calcium phosphate salts, including hydroxyapatite, but can also consist of calcium oxalates and octacalcium phosphate as seen in e.g., kidney stones.

Tissues affected by ectopic calcification often show evidence of tissue alteration and/or necrosis. Indeed, ectopic calcification is frequently observed in soft tissues as a result of injury, disease, and aging. However, ectopic calcifications are not only associated with cell death. For example, ectopic calcifications may occur in native aortic valve stenosis or in the Monckeberg's type calcification that is seen in blood vessels from diabetic and uremic patients. Although most soft tissues can undergo calcification, skin, kidney, tendons, and cardiovascular tissues appear particularly prone to developing this pathology. In addition, a number of prosthetic devices such as artificial heart valves, are prone to ectopic calcification.

Ectopic calcification can lead to clinical symptoms when it occurs in cardiovascular tissues, particularly when it occurs in arteries and heart valves. In arteries, calcification is correlated with atherosclerotic plaque burden and increased risk of myocardial infarction, increased ischemic episodes in peripheral vascular disease, and increased risk of dissection following angioplasty. In the heart, valves are particularly prone to calcification. Indeed, calcific aortic stenosis is a rather common condition, occurring in approximately 1-2% of the elderly population. Calcific aortic stenosis is characterized by stiffening, tearing, and mechanical failure of the valve. Common conditions such as congenital anomalies, rheumatic fever, inflammatory changes, renal disease, and age are all risk factors for calcific aortic valve stenosis.

Treatment for severe symptomatic calcific aortic stenosis is aortic valve replacement. Indeed, more than 40,000 patients undergo valve replacement each year in the United States alone (see e.g., O'Keefe, J. H., et al. (1991) Postgrad. Med. 89:143). Although valve replacement has resulted in dramatic improvement in longevity and symptoms of patients with valve disease, an important cause for the failure of artificial heart valves is, in fact, calcification of the prosthetic valve.

Also included in the disclosure are methods for treating other autoimmune diseases such as Rheumatoid Arthritis (RA).

RA is an inflammatory condition where articular cartilage of affected joints is being degraded by an active process involving cells of the immune system as well as the tissues of the joint (i.e., the synovial membrane, the cartilage and subchondral bone). The etiology of RA is complex and a number of environmental and genetic factors have been suggested a role in the development of the disease.

Studies in humans and animal models of OA and RA have demonstrated a progressive depletion of articular cartilage matrix macromolecules as the disease develops. In RA the cartilage degradation tends to occur more rapidly due to the potent catabolic stimuli evoked by the active inflammatory response and immune system components mediating the tissue destruction in the disease. The progression of joint destruction varies widely between individual patients with a marked cyclical pattern characterized by periods of elevated disease activity (flare ups) intermittent with more ‘silent’ periods. This cyclical pattern of disease activity is prominent for RA, as well as osteoarthritis and multiple sclerosis.

The most commonly used drugs for the treatment of RA are NSAIDs and Opioids used for treating the pain and symptoms of the patients and disease modifying anti-rheumatic drugs (DMARD's) and corticosteroids as well as more specific anti-inflammatory agents such as TNF-αt or IL-1 antagonists. In OA treatment, NSAID and DMARD's also play an important role. The use of NSAIDs and simple analgesics e.g., paracetamol, have been shown to reduce the pain of OA. In addition topical NSAIDs can provide some pain relief and are associated with fewer side effects than the systemic drug treatments. Intra-articular steroid injections can be used for inflammatory flares, but in established OA the effects are short-lived, In RA better effects have been obtained with systemic as well as intra-articular steroid administration, and this remains one of the most common treatment options for the disease, in spite of the adverse effects associated with long term steroid use such as accelerated systemic bone loss leading to osteoporosis and an increased risk of fragility fracture. In more advanced cases of OA, hip and knee replacements are an effective surgical option for relieving pain and improving function.

The aim of current therapies of these diseases is mainly to relieve pain and disease symptoms. NSAID, opioids and DMARD's have proved effectiveness in relieving the symptoms of OA and RA but their effect on decreasing cartilage catabolism has not been well documented. Some of them, like sodium salicylate, have shown inhibiting properties of the proteoglycan synthesis which may jeopardize the cartilage repair process. Other drugs, such as tiaprofenic acid, which do not inhibit the proteoglycan synthesis, have shown in vitro that they are able to decrease OA cartilage catabolism, (Pelletier et al. J of Rheumatology 1989; 16:5, 646-655). However, they have been unable to provide any significant protective effect in development of OA when administrated to patients suffering from the latter, (Edward C. Huskisson et al. J Rheumatol 1995; 22:10-1941-1946). Doxycycline, a member of the tetracycline family, was also shown to reduce, in vivo, the severity of OA lesions in the dog anterior cruciate ligament trans-section (ACL) model of joint damage induced OA while reducing metalloprotease activity, (Yu et al. Arthr Rheum 35:1150-1159, 1992). Recent data suggests that the action of corticosteroids is associated with a reduction in the synthesis of the cartilage matrix degrading MMP, stromelysin-1 by chondrocytes. (see: Pelletier et al., J Arthr Rheum 37:414-423, 1994; and Pelletier et al., J Lab Invest 72:578-586, 1995).

In the clinical management of OA, the focus of medical intervention has been to relieve disease symptoms (i.e., by using Non-Steroidal-Anti-Inflammatory Drugs (NSAID) and the newer COX-2 inhibitors). None of the drugs in current clinical use, with the exception of glucosamine sulphate has demonstrated significant effects to halt the underlying tissue destruction (i.e., articular cartilage thinning and subchondral bone changes) (Christgau et al. Clin Exp Rheumatol. 2004; 22: 36-42). It is very questionable if the palliative agents used in the clinical management of OA have any structure modifying effects. In fact, recent reviews of the literature indicates that different classes of NSAIDs may have effects on chondrocytes ranging from deleterious to beneficial with regard to glycosaminoglycan synthesis. Very few, if any, therapies are available that has a convincing effect of slowing or halting the underlying cartilage degradation, which is the prime culprit causing the progressive joint destruction accompanying the disease. Thus, there is an unmet therapeutic need for compounds, which can act on the cells and enzyme systems mediating the cartilage degradation in OA.

In the management of acute and chronic pain in joint diseases such as RA and OA, the ability to prevent the onset of pain, lessen its intensity, and interfere with the development of sensitization contributing to hyperalgesia for days following traumatic pain can greatly benefit the patient as pain represents the main clinical symptoms. Accordingly the palliative treatment is important and effective management of the joint diseases. In situations where pain can be anticipated, i.e., at the early clinical signs of a flare up in disease activity, the NSAID may be optimized by administration of elevated doses and continuing to dose the NSAID on a regular schedule to minimize pain and inflammation. Patients benefit from receiving optimal NSAID doses, and in some cases very high doses of these palliative agents are required to efficiently relieve the pain. In conditions of chronic pain, the dosing of palliative agents are of paramount importance, and as RA and OA patients are likely to receive the drugs over long periods of time due to the chronic nature of the diseases, the side effects of these interventions becomes of paramount importance. This represents a major problem in current clinical practice as most NSAID's and other analgesic agents are associated with severe gastro intestinal side effects.

Therefore, the present disclosure also relates to the use of antibodies to osteopontin, to selectively block osteopontin's role in enhancing cell mediated autoimmunity that occurs in a acute and chronic diseases, including RA and OA.

### Pharmaceutical Formulations

Pharmaceutical formulations comprising an antibody, identified by the methods of the present disclosure can be prepared for storage by mixing the protein having the desired degree of purity with optional physiologically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN®, PLURONICS® or polyethylene glycol (PEG).

The formulation described herein can also contain more than one active compound as necessary for the particular indication being treated. Such molecules are suitably present in combination in amounts that are effective for the purpose intended.

In one embodiment, the pharmaceutical formulations can comprise an antibody identified by the methods described herein. In certain embodiments, the pharmaceutical formulation can be in a microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

In still other embodiments, sustained-release preparations can be prepared. Suitable examples of sustained-release preparations include semi-permeable matrices of solid hydrophobic polymers containing the protein, which matrices are in the form of shaped articles, e.g., films, or microcapsule. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and .gamma. ethyl-L-glutamate, non-degradable ethylene-vinyl acetate, degradable lactic acid-glycolic acid copolymers such as the Lupron Depot® (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated proteins remain in the body for a long time, they can denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization can be achieved by modifying sulfhydryl residues, lyophilizing from acidic solutions, controlling moisture content, using appropriate additives, and developing specific polymer matrix compositions.

### Non-Therapeutic Uses

The antibodies, identified by the methods of the present disclosure can be used non-therapeutic agents, for example, as affinity purification agents, diagnostic reagents and biomarkers for disease progression. In such an embodiment, a protein of interest is immobilized on a solid phase such a Sephadex resin or filter paper, using methods well known in the art. The immobilized protein is contacted with a sample containing the target of interest (or fragment thereof) to be purified, and thereafter the support is washed with a suitable solvent that will remove substantially all the material in the sample except the target protein, which is bound to the immobilized antibody. Finally, the support is washed with another suitable solvent, such as glycine buffer, pH 5.0, which will release the target protein.

Antibodies identified by the methods of the present disclosure can also be useful in diagnostic assays for the targeted protein, e.g., detecting its expression in specific cells, tissues, or serum. Such diagnostic methods can be useful in cancer diagnosis.

For diagnostic applications, the proteins will typically be labeled with a detectable moiety. In certain embodiments, the detectable moiety can be selected from the following categories: (a) Radioisotopes, such as ³⁵S, ¹⁴C, ¹²⁵I, ³H, and ¹³¹I. The antibody can be labeled with the radioisotope using the techniques described in Current Protocols in Immunology, Volumes 1 and 2, Coligen et al., Ed. Wiley-Interscience, New York, N.Y., Pubs. (1991) for example and radioactivity can be measured using scintillation counting; (b) Fluorescent labels such as rare earth chelates (europium chelates) or fluorescein and its derivatives, rhodamine and its derivatives, dansyl, Lissamine, phycoerythrin and Texas Red are available; (c) enzyme-substrate labels.

Various enzyme substrate labels are known in the art and U.S. Pat. No. 4,275,149 provides a review of some of these. The enzyme generally catalyzes a chemical alteration of the chromogenic substrate which can be measured using various techniques. For example, the enzyme can catalyze a color change in a substrate, which can be measured spectrophotometrically. Alternatively, the enzyme can alter the fluorescence or chemiluminescence of the substrate. Techniques for quantifying a change in fluorescence are described above. The chemiluminescent substrate becomes electronically excited by a chemical reaction and can then emit light which can be measured (using a chemiluminometer, for example) or donates energy to a fluorescent acceptor. Examples of enzymatic labels include luciferases (e.g., firefly luciferase and bacterial luciferase; U.S. Pat. No. 4,737,456), luciferin, 2,3-dihydrophthalazinediones, malate dehydrogenase, urease, peroxidase such as horseradish peroxidase (HRPO), alkaline phosphatase, β-galactosidase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like. Techniques for conjugating enzymes to antibodies are described in O'Sullivan et al., Methods for the Preparation of Enzyme-Antibody Conjugates for use in Enzyme Immunoassay, in Methods in Enzymol. (ed J. Langone & H. Van Vunakis), Academic press, New York, 73:147 166 (1981).

In certain embodiments, enzyme-substrate combinations can include, for example: (i) Horseradish peroxidase (HRPO) with hydrogen peroxidase as a substrate, wherein the hydrogen peroxidase oxidizes a dye precursor (e.g., orthophenylene diamine (OPD)); (ii) alkaline phosphatase (AP) with para-Nitrophenyl phosphate as chromogenic substrate; and (iii) β-D-galactosidase (β-D-Gal) with a chromogenic substrate (e.g., p-nitrophenyl-β-D-galactosidase) or fluorogenic substrate 4-methylumbelliferyl-β-D-galactosidase.

Numerous other enzyme-substrate combinations are available to those skilled in the art. For a general review of these, see U.S. Pat. Nos. 4,275,149 and 4,318,980.

The antibodies identified by the methods of the present disclosure can be employed in any known assay method, such as competitive binding assays, direct and indirect sandwich assays, and immunoprecipitation assays. Zola, Monoclonal Antibodies: A Manual of Techniques, pp. 147 158 (CRC Press, Inc. 1987).

The antibodies can also be used for in vivo diagnostic assays. Generally, the antibody is labeled with a radio nuclide (such as ¹¹¹In, ¹⁴C, ¹³¹I, ¹²⁵I, ³H, ³²P or ³⁵S) so that the tumor can be localized using immunoscintiography.

### EXAMPLES

### Example 1 Immortalization of Cells and Initial Screening of Cells

Frozen peripheral blood mononuclear cells (PBMCs) are thawed and stained with directly labeled antibodies to CD22 (Pharmingen, BD Biosciences, US) and to immunoglobulin (Ig) M, IgD, and IgA (Jackson ImmunoResearch, US). CD22+ IgM-, IgD-, IgA-B cells are isolated using a FACS-Aria (Becton Dickinson) and immortalized at 30 B cells/well in replicate cultures using EBV in the presence of CpG oligodeoxynucleotide 2006 (Mycrosynth, CH) and irradiated allogeneic PBMC, as previously described (Traggiai et al., (2004) Nat. Med. 10 871-875). Cells are cultured in complete RPMI 1640 supplemented with 10% fetal calf serum (HyClone Laboratories, US). Culture supernatants are harvested after 14 days and assayed for neutralizing activity against osteopontin via ELISA assay as described previously (Sakata et al. (2001) J. Rheumatol. 28 1492-1495; Du et al. (2005) Rheumatol. Int. 26 35-41) using purified recombinant human osteopontin (Chemicon International, Temecula, Calif., USA). Cultures with measurable neutralizing activity are cloned at 0.5 cell/well in the presence of CpG 2006 and irradiated PBMCs. B cell clones are cultured at a high cell density in complete RPMI 1640 10% Ig-depleted fetal calf serum to produce enriched supernatants containing 1-3 mg secreted monoclonal antibodies/ml. MAbs are purified on protein G columns (GE Healthcare Europe). The isotype, subclass, and light chain of the mAbs is characterized by ELISA using specific antibodies and HRP-labeled anti-human Ig antibody (Southern Biotechnology, US). Antibodies are quantified with reference to a standard certified preparation (Sigma-Aldrich, US).

### Example 2 Epitope Mapping

OPN-related fragment peptides (CVDTYDGRGDSVVYGLRS (C+V153 to S169); KSKKFRRPDIQYPDATDEC (K170 TO E187+C), IPVKQADSGSSEEKQC (117 to Q31+C) and SKEEDKHLKFRISHELDSASSEVNC (S290-N340+C) are prepared via standard solid phase synthesis and diluted with 0.1 M carbonate buffer, pH 9.5 to 10 pg/ml, and are immobilized at 50 μl/well on a 96-well plate.

After rinsing with PBS and blocking with 0.1% BSA/PBS/0.05% NaN₃ solution, a 2-fold dilution series of the 100-fold dilution of donor anti-sera is placed at 50 μl in a well, for reaction at 37° C. for 30 minutes.

After termination of the reaction, the wells are rinsed four times with 0.05% Tween 20-PBS. Then, 50 μl each of HRP-labeled anti-rabbit IgG (manufactured by IBL Co., Ltd.) is added to each well, for reaction at 37° C. for 30 minutes. After termination of the reaction, 100 μl each of 0.05 M citrate buffer, pH 4.5 containing 0.4 mg/ml orthophenylenediamine (OPD) and aqueous 0.03% hydrogen peroxide is added to each well. Then, the plate is left to stand in darkness at ambient temperature for 15 minutes, to complete the chromogenic reaction. After the chromogenic reaction, 100 μl of 1N sulfuric acid is added to each well, to terminate the reaction, and the absorbance at 492 nm can be determined.

### Example 3 Screening of Antibodies for Inhibition of Osteopontin/Receptor Binding

Secreted osteopontin has been shown to bind to two primary cell-surface receptors: the hyaluronic acid receptor (CD44) and members of the integrin family (including αvβ1, αvβ3, αvβ5, α4β1, α5β1, α8β1, α9β1) (Scatena et al. (2007) Arterioscler. Thromb. Vasc. Biol. 27: 2302-2309). Osteopontin binding to the integrins is thought to occur through an arginine-glycine-aspartate-(RGD)-containing domain, as well as a cryptic SVVYGLR (SLAYGLR in mice and rats) which becomes exposed upon thrombin cleavage. An ELVTDFTDLPAT domain is also thought to facilitate binding of osteopontin to certain integrins (Scatena et al. (2007) Arterioscler. Thromb. Vasc. Biol. 27: 2302-2309). Monoclonal antibodies which prevent osteopontin binding to CD44 or integrin receptors could be identified as follows: 1) purified extracellular domains of either CD44 and/or the various integrins could be coated onto wells of a microtiter plate. A purified epitope-tagged form of osteopontin could subsequently be incubated with these receptor extracellular domains in the presence or absence of candidate anti-osteopontin antibodies. Bound osteopontin could be detected by ELISA on the basis of immunodetection of the specific epitope sequence fused to osteopontin. 2) Alternatively, CD44 and/or the various integrins could be expressed in cells (either in full-length form or as extracellular domains tethered to the cell membrane (e.g., by a glycosylphosphatidylinositol moiety). The CD44-, integrin-expressing cells could be cultured in a microtiter plate, and then a purified epitope-tagged osteopontin could be incubated in the presence or absence of candidate anti-osteopontin monoclonal antibodies. Detection of bound epitope-tagged osteopontin would be accomplished similarly by ELISA using an antibody which recognizes the epitope tag.

### Example 4 Screening of Antibodies for Inhibition of Cell Adhesion

Cell adhesion, one assay which could be used to assess the neutralizing capability of the monocional antibodies against osteopontin, may be performed as previously described (Hu et al. (1995) 270: 9917-9925). HEK-293 cells, or other cell-types, are suspended in buffer consisting of Hank's balanced salt solution, 50 mM Hepes pH 7.4, 0.5 mM Mn2+, and 1 mg/mL bovine serum albumin, and applied to wells of a microtiter plate containing recombinant human osteopontin in the presence or absence of candidate monoclonal anti-osteopontin antibodies for 90 minutes at 37° C. In certain cases, cells may be first transfected with cDNAs encoding various integrins, including but not limited to αᵥβ family. Microtiter wells coated with osteopontin overnight at 4° C. are blocked with 30 mg/mL BSA in TBS at 37° C. Subsequently, cells are incubated in the osteopontin-coated wells for 45 minutes at 37° C., followed by wash with TBS and gentle aspiration to remove non-adherent cells. Adherent cells could be determined colorimetrically by measuring lysosomal acid phosphatase activity.

### Example 5 Inhibitory Activity of Anti-Osteopontin Monoclonal Antibodies on Downstream Cytokine Production

Previous studies have demonstrated that osteopontin can up-regulate various cytokines, including macrophage chemoattractant protein 1 (MCP1) and macrophage inflammatory protein 1β (MIP-1β) (Zheng et al. (2009) 60: 1957-1965). As described in Zheng et al., peripheral blood mononuclear cells (PBMCs) cultured in RPMI 1640 in microtiter plates are stimulated with osteopontin in the presence or absence of candidate anti-osteopontin monoclonal antibodies. OPN-induced cytokines (e.g., MCP1 and MIP-1β) are measured by ELISA.

### Example 6 Inhibitory Activity of Human Auto-Antibodies on Human Peripheral Leukocyte Migration

By the following method, the inhibitory activity of the human antibodies on cytokine-activated human peripheral leukocyte migration can be examined.

By the Ficoll method, a monocyte fraction and a neutrophil fraction are separated from normal human peripheral blood (P. M. Daftarian et al., (1996): Journal of Immunology, 157, 12-20). The intermediate layer between Ficoll and serum is collected and cultured in a flask at 37° C. for one hour. The resulting attached cells are used as monocyte. To the erythrocyte layer remaining after collection of the monocyte fraction is added a 5-fold volume of 3% dextran-PBS to aggregate erythrocyte, followed by centrifugation at 150×g and 4° C. for 5 minutes.

The aggregated erythrocyte is precipitated, while in the resulting supernatant, neutrophil exist in suspended state. Then, the fraction is centrifuged at 500×g and ambient temperature for 20 minutes, to recover neutrophil. The monocyte and neutrophil as recovered in such manner is cultured overnight with human TNF-α (20 ng/mL) for activation. Then, the resulting activated monocyte and neutrophil is used for migration experiments.

The migration experiments are done, using a 48-well micro chemotaxis chamber (manufactured by Neuro Probe Inc.). After various concentrations of the human antibody is added to the thrombin-cleaved OPN and were then preliminarily left to stand at 37° C. for 15 minutes, the mixtures are added to the lower chamber (to a final human OPN concentration of 10 pg/mL). Placing thereon a polycarbonate filter (pore size of 5 μm), further, a cell suspension (2×10⁶ cells/mL) of 50 μL is added to the upper chamber.

After culturing in the presence of 5% CO₂ at 37° C. for 2 hours, the polycarbonate filter is removed to discard the cells on the upper surface of the filter; subsequently, the cells infiltrating to the back face of the filter are stained with Diff-Quick (manufactured by Baxter International Inc.). The stained cells are counted at a magnification×40. The results are shown as mean cell counts (cells/mm³)±SD in 6 wells.

We claim:

1. A method for selecting a therapeutic antibody to osteopontin comprising the steps of;
a) screening a subject with osteoarthritis, multiple sclerosis or rheumatoid arthritis, for osteopontin specific antibodies, and;
b) isolating or amplifying one or more nucleic acids from the patient's B cells encoding an antibody heavy or light chain, or portion thereof, that binds to osteopontin.

2. The method of claim 1, wherein the one or more nucleic acids obtained from the subject's B cells is obtained by PCR amplification of isolated B cells.

3. The method of claim 1, wherein the isolated B cells are immortalized.

4. The method of claim 1, wherein the subject is a human donor.

5. The method of claim 4, wherein, the human donor is pre-selected to have antibody titers for osteopontin binding of greater than (>1:1000) measured via an ELISA assay.

6. The method of claim 4, wherein the human donor has multiple sclerosis.

7. The method of claim 4, wherein the human donor has osteoarthritis.

8. The method of claim 4, wherein the human donor has rheumatoid arthritis

9. The method of claim 4, wherein the human donor is in stable remission.

10. The method of any of claims 1-9, wherein, the antibodies are screened for binding to an epitope within an arginine-glycine-aspartate (RGD) containing domain of osteopontin.

11. The method of any of claims 1-9, wherein, the antibodies are screened for binding to an epitope within a serine-valine-valine-tyrosine-glycine-leucine-arginine (SVVYGLR) containing domain of osteopontin.

12. The method of claims 1-9, wherein, the antibodies are screened for binding to an epitope within the ELVTDFTDLPAT containing domain of osteopontin.

13. The method of claims 1-9, wherein, the antibodies are screened for selective binding to OPN-a.

14. The method of claims 1-9, wherein, the antibodies are screened for binding to OPN-b.

15. The method of claims 1-9, wherein, the antibodies are screened for binding to OPN-c.

16. The method of claims 1-9, wherein, the antibodies are screened for binding to a combination of two of more of OPN-a, OPN-b, and OPN-c.

17. The method of claims 1-9, wherein, the antibodies are screened for binding to osteopontin-R.

18. The method of claims 1-9, wherein, the antibodies are screened for binding to osteopontin-L.

19. The method of claims 1-9, wherein, the antibodies are screened for binding to osteopontin-166.

20. The method of claims 1-9, wherein, the antibodies are screened for binding to a CD44 binding site of osteopontin.

21. The method of claims 1-9, wherein, the antibodies are screened for binding to an αᵥβ₃ binding site of osteopontin.

22. The method of claims 1-9, wherein, the antibodies are screened for binding to an αᵥβ₁ binding site of osteopontin.

23. The method of claims 1-9, wherein, the antibodies are screened for binding to an αᵥβ₅ binding site of osteopontin.

24. The method of claims 1-9, wherein, the antibodies are screened for binding to an α₅β₁ binding site of osteopontin.

25. The method of claims 1-9, wherein, the antibodies are screened for binding to both an αᵥβ₃ binding site of osteopontin and a CD44 binding site osteopontin.

26. A method for selecting a human therapeutic antibody to osteopontin comprising the steps of;
a) isolating memory B cells obtained from a human donor with osteoarthritis, multiple sclerosis, or rheumatoid arthritis;
b) immortalizing the cells isolated in step a) to produce memory B cell cultures;
c) screening the memory B cell cultures obtained in step b) for cultures producing osteopontin specific antibodies;
d) isolating or amplifying one or more nucleic acids from the memory B cell cultures producing osteopontin specific antibodies of step c) encoding an antibody heavy or light chain, or portion thereof, that binds to osteopontin.

27. A method for selecting a human therapeutic antibody for treating multiple sclerosis comprising the steps of;
a) isolating memory B cells obtained a human donor with osteoarthritis, multiple sclerosis, or rheumatoid arthritis;
b) immortalizing the cells isolated in step a) to produce memory B cell cultures;
c) screening the memory B cell cultures obtained in step b) for cultures producing osteopontin specific antibodies;
d) isolating or amplifying one or more nucleic acids from the memory B cell cultures producing osteopontin specific antibodies of step c) encoding an antibody heavy or light chain, or portion thereof, that binds to osteopontin.

28. A method for selecting a human therapeutic antibody for treating rheumatoid arthritis, comprising the steps of;
a) isolating memory B cells obtained from a human donor with osteoarthritis, multiple sclerosis, or rheumatoid arthritis;
b) immortalizing the cells isolated in step a) to produce memory B cell cultures;
c) screening the memory B cell cultures obtained in step b) for cultures producing osteopontin specific antibodies;
d) isolating or amplifying one or more nucleic acids from the memory B cell cultures producing osteopontin specific antibodies of step c) encoding an antibody heavy or light chain, or portion thereof, that binds to osteopontin.

29. A method for selecting a human therapeutic antibody for treating osteoarthritis comprising the steps of;
a) isolating memory B cells obtained from a human donor with osteoarthritis, multiple sclerosis, or rheumatoid arthritis;
b) immortalizing the cells isolated in step a) to produce memory B cell cultures;
c) screening the memory B cell cultures obtained in step b) for cultures producing osteopontin specific antibodies;
d) isolating or amplifying one or more nucleic acids from the memory B cell cultures producing osteopontin specific antibodies of step c) encoding an antibody heavy or light chain, or portion thereof, that binds to osteopontin.

30. A process for preparing memory B cell clones expressing a therapeutic antibody to osteopontin, to treat an osteopontin related disease comprising the steps of;
a) isolating memory B cells obtained from a human donor with osteoarthritis, multiple sclerosis, or rheumatoid arthritis;
b) isolating a sample of memory B cells from the human donor,
c) immortalizing the memory B cells by transforming memory B cells with Epstein Barr virus (EBV) in the presence of a polyclonal B cell activator;
d) screening the memory B cells for the expression of antibodies that bind to osteopontin, and selecting memory B cells that exhibit antibodies that specifically bind to osteopontin.

31. A method for treating an osteopontin related disease, in a patient in need thereof, comprising the steps of:
a) identifying one or more human donors suffering from osteoarthritis, multiple sclerosis or rheumatoid arthritis; wherein the human donors are characterized by the presence of anti-osteopontin antibodies; and
b) isolating B cells from the positive human donor patients screened in step a);
c) isolating or amplifying an nucleic acid from the selected B cell of step b) encoding an antibody heavy or light chain, or portion thereof, that binds to osteopontin,
d) administering the antibody to osteopontin encoded by the nucleic acid obtained in step (c) to the patient.

32. The method of any of claims 26-31, wherein, the human donor is pre-selected to have antibody titers for osteopontin binding of greater than (>1:1000) measured via an ELISA assay

33. The method of any of claims 26-31, wherein the human donor has multiple sclerosis

34. The method of any of claims 26-31, wherein the human donor has osteoarthritis.

35. The method of any of claims 26-31, wherein the human donor has rheumatoid arthritis

36. The method of any of claims 26-35, wherein the human donor is in stable remission.

37. The method of any of claims 26-36, wherein, the antibodies are screened for binding to an epitope within an arginine-glycine-aspartate (RGD) containing domain of osteopontin.

38. The method of any of claims 26-36, wherein, the antibodies that screened for binding to an epitope within a serine-valine-valine-tyrosine-glycine-leucine-arginine (SVVYGLR) containing domain of osteopontin.

39. The method of any of claims 26-36, wherein, the antibodies that screened for binding to an epitope within the ELVTDFTDLPAT containing domain of osteopontin.

40. The method of any of claims 26-36, wherein, the antibodies are screened for binding to OPN-a.

41. The method of any of claims 26-36, wherein, the antibodies are screened for binding to OPN-b.

42. The method of any of claims 26-36, wherein, the antibodies are screened for binding to OPN-C.

43. The method of any of claims 26-36, wherein, the antibodies are screened for binding to any combination of OPN-a, OPN-b, and OPN-c.

44. The method of any of claims 26-36, wherein, the antibodies are screened for binding to osteopontin-R.

45. The method of any of claims 26-36, wherein, the antibodies are screened for binding to osteopontin-L.

46. The method of any of claims 26-36, wherein, the antibodies are screened for binding to osteopontin-166.

47. The method of any of claims 26-36, wherein, the antibodies are screened for binding to a CD44 binding site of osteopontin.

48. The method of any of claims 26-36, wherein, the antibodies are screened for binding to an αᵥβ₃ binding site of osteopontin.

49. The method of any of claims 26-36, wherein, the antibodies are screened for binding to an αᵥβ₁ binding site of osteopontin.

50. The method of any of claims 26-36, wherein, the antibodies are screened for binding to an αᵥβ₅ binding site of osteopontin.

51. The method of any of claims 26-36, wherein, the antibodies are screened for binding to an α₅β₁ binding site of osteopontin.

52. The method of any of claims 26-36, wherein, the antibodies are screened for binding to both an αvβ3 binding site of osteopontin and a CD44 binding site osteopontin.

53. The method of claim 31, wherein the osteopontin related disease is selected from the group consisting of multiple sclerosis, rheumatoid arthritis, osteoarthritis, metastatic cancer, systematic lupus erythematosis or autoimmune renal disease, idiopathic fibrosis, allergic disease, hepatitis, valvular heart disease, cardiac remodeling, and ectopic tissue calcification.

54. An antibody made by a method of any one of claims 1 to 24.

55. A purified human therapeutic auto antibody to osteopontin.

56. A pharmaceutical composition comprising an antibody of claims 54 or 55.

57. The antibody of any of claims 54 to 55, wherein, the antibody binds selectively to OPN-a.

58. The antibody of any of claims 54-55, wherein, the antibody binds selectively to OPN-b.

59. The antibody of any of claims 54-55, wherein, the antibody binds selectively to OPN-c.

60. The antibody of any of claims 54-55, wherein, the antibody binds selectively to any combination of OPN-a, OPN-b, and OPN-c.

61. The antibody of any of claims 54 to 55, wherein, the antibody binds to osteopontin-R.

62. The antibody of any of claims 54 to 55 wherein, the antibody binds to osteopontin-L.

63. The antibody of any of claims 54 to 55, wherein, the antibody binds to osteopontin-166.

64. The antibody of any of claims 54 to 55, wherein, the antibody binds to a CD44 binding site of osteopontin.

65. The antibody of any of claims 54 to 55, wherein, the antibody binds to an αᵥβ₃ binding site of osteopontin.

66. The antibody of any of claims 54 to 55, wherein, the antibody binds to an αᵥβ₁ binding site of osteopontin.

67. The antibody of any of claims 54 to 55 wherein, the antibody binds to an αᵥβ₅ binding site of osteopontin.

68. The antibody of any of claims 54 to 55, wherein, the antibody binds to an α₅β₁ binding site of osteopontin.

69. The antibody of any of claims 54 to 55, wherein, the antibody binds to both an αᵥβ₃ binding site of osteopontin and a CD44 binding site osteopontin.

70. The antibody of any of claims 54 to 55, wherein, the antibody is a bispecific antibody.

71. The antibody of any of claims 54 to 55, wherein, the antibody is a humanized antibody.
